(19) United States
(12) Patent Application Publication
(10) Pub. No.: US 2026/0345935 A1
(43) Pub. Date: Nov. 5, 2026

(54) INTUBATION SYSTEM COMPRISING A TUBE LOCKING DEVICE, LARYNGOSCOPE AND TUBE

(71) Applicant: KARL STORZ SE & Co. KG, Tuttlingen (DE)

(72) Inventors: Felix VOGT, Tuttlingen (DE); Andreas EFINGER, Tuttlingen (DE)

(21) Appl. No.: 19/486,570

(22) PCT Filed: May 16, 2024
(86) PCT No.: PCT/EP2024/063593

§ 371 Date: Nov. 21, 2025

(30) Foreign Application Priority Data
May 22, 2023 (DE) 10 2023 113 304.7

(51) Int. Cl. A61M 16/04; A61B 1/00; A61B 1/267

(52) U.S. Cl. CPC A61M 16/0434; A61B 1/00128; A61B 1/267; A61M 16/0418; A61M 2210/1032

(57) ABSTRACT

The invention relates to an intubation system (100, 1000, 2000, 3000, 4000, 5000, 6000, 7000, 8000, 9000), comprising a tube (102, 1002, 7002), a stylet (104), wherein the tube (102, 1002, 7002) can be at least partially pushed onto the stylet (104), a laryngoscope (106, 8006), comprising a spatula (108, 2008, 8008), wherein for insertion the stylet (104) can be pushed forward, together with the tube (102, 1002, 7002) which is pushed onto the stylet (104), relative to the spatula (108, 2008, 8008), and wherein the stylet (104), after insertion, can be pulled out of the tube (102, 1002, 7002), and a tube locking device (110, 1010, 2010, 3010, 4010, 5010, 6010, 7010, 8010, 9010), which is designed to prevent the tube (102, 1002, 7002) from being pulled back relative to the spatula (108, 2008, 8008) when the stylet (104), after having been pushed forward, is pulled back out of the tube (102, 1002, 7002).

The present invention relates to an intubation system, a laryngoscope for an intubation system, and a tube for an intubation system.

If a patient's respiratory function is impaired due to a medical emergency such as an accident or illness, or if the patient's own breathing is artificially restricted for the purpose of performing an operation, it may be necessary to take measures to secure the airway. The gold standard for securing the airway is endotracheal intubation. For endotracheal intubation, a user typically inserts an endotracheal tube partially into the trachea (windpipe) through the patient's mouth. To do this, the user inserts the tube between the vocal cords of the larynx into the trachea and positions a distal end of the tube in a desired position within the trachea. The user then holds the tube in position in order to be able to immobilize it. A cuff is attached to the tube just above the distal end and is inflated in order to immobilize the tube. Besides immobilizing the tube, this also seals the trachea and reduces the risk of aspiration. A ventilation means such as a ventilator or a resuscitation bag is then provided at a proximal end of the tube in order to enable ventilation of the patient.

Although endotracheal intubation is the gold standard for securing the airway, it poses major problems for many doctors. There is therefore great interest in aids for simplifying intubation. Furthermore, complicated intubations may occur specific to the patient or the situation. For example, a patient may be bleeding, or the larynx may not be readily visible. In order to be able to secure the airway as quickly as possible in an emergency situation, aids are provided.

One common aid for intubation is a laryngoscope. By means of the laryngoscope, the larynx can be visualized to facilitate intubation. The laryngoscope usually comprises a handle and a spatula. The user can use the spatula to compress soft tissues in the floor of the mouth, push down the lower jaw, and move the tongue to the side. This gives the user a direct view toward the larynx and allows him to see the vocal cords, for example. Often a light source and a camera are also provided on the spatula. In this case, the larynx can be imaged and displayed on a monitor, thereby further simplifying intubation.

Various types of laryngoscope have been developed over time. In principle, a distinction can be made between laryngoscopes that are intended for single use and those that can be used multiple times. Furthermore, the shape of the spatula can differ. On the one hand there are laryngoscopes that have a straight spatula, and on the other there are laryngoscopes that have a curved spatula. In the case of laryngoscopes that have a straight spatula, pressure is applied to the epiglottis using the spatula, and the epiglottis is urged in the direction of the handle in order to provide a view of the glottis. However, in adults, the upper incisors can easily be broken when the spatula is inserted into the mouth. This is one of the reasons why laryngoscopes of this type are more commonly used in newborns and infants. In adults, laryngoscopes that have a curved spatula have become the standard. These conform to the tongue better and follow the shape of the throat. The spatula is passed in front of the epiglottis, and the laryngoscope is pulled in the direction of the handle of the laryngoscope. This causes the epiglottis to rise and provides a view of the glottis. Spatulas are generally available in different sizes, which are used depending upon the patient's anatomy. This means that, for example, an ambulance must have many different spatulas and/or laryngoscopes available.

Another common aid is a guide rod, also called a stylet. This is inserted into the tube to give it shape and stability, which makes it easier to insert the tube into the airway. As soon as the stylet is in position in the tube, the tube can be inserted through the vocal cords and into the trachea with the aid of the stylet. There are different types of stylet, including rigid stylets and flexible stylets. The rigid stylet is a straight wire, often made of metal or plastic, while the flexible stylet is made of a flexible plastic that can easily bend in order to follow the anatomical course of the airways. In addition, there are flexible stylets which have a distal portion that can be bent by a user by way of operating elements. The flexible stylet is often used in children and in patients with difficult anatomical conditions. In addition, a camera may be provided on the stylet in order to image the patient's airways. Such a stylet is also called a video stylet and is a common aid, particularly in difficult intubations. By means of the video stylet, the tube can also be more easily positioned at the desired position in the trachea, since a side wall of the trachea can be viewed, and the tube can be positioned relative to anatomical structures.

Once the tube has been inserted by means of the stylet, the stylet must be pulled back out of the tube so that the patient can be ventilated. It is important that the tube remain in the desired position, and in particular not pulled back out of the trachea. Therefore, in order to be able to safely pull the stylet back out of the tube, the tube must be immobilized relative to the stylet. For example, a second user may pull out the stylet, while the other user immobilizes the laryngoscope with one hand and the tube with the other hand.

Particularly in emergency situations and/or in confined spaces, such as in an ambulance, it may be difficult to use the second user to pull out the stylet. In addition, coordination between different users is inherently complicated and often results in a lower quality of handling. Furthermore, the costs of intubation may increase as a result, since more trained personnel must be available.

Generally, the tube can usually move relative to the spatula. A single user may therefore find it difficult to keep the tube in position.

Proceeding from the prior art, the object of the invention is to provide an intubation system with a high degree of operational reliability and efficiency.

This object is achieved according to the invention by an intubation system, a laryngoscope, and a tube, as described herein and defined in the claims.

The present invention provides an intubation system, comprising a tube, a stylet, wherein the tube can be at least partially pushed onto the stylet, a laryngoscope, comprising a spatula, wherein, for insertion, the stylet can be pushed forward, together with the tube which is pushed onto the stylet, relative to the spatula, and wherein the stylet, after insertion, can be pulled out of the tube, and a tube locking device, which is designed to prevent the tube from being pulled back relative to the spatula when the stylet, after having been pushed forward, is pulled back out of the tube.

In addition, a laryngoscope for an intubation system according to the invention is provided.

Furthermore, a tube for an intubation system according to the invention is provided.

The features according to the invention make it possible to provide an intubation system with a high degree of operational reliability and efficiency. Advantageously, the intubation system can also be operated and/or handled safely and reliably by a single user. Furthermore, the tube can be prevented from moving out of position within the trachea when the stylet is being pulled out. In particular, the risk of the tube being completely pulled out of the trachea is reduced. In particular, the user can be assisted in performing complicated intubations. Furthermore, operation of the intubation system is more cost-effective and/or efficient. In principle, fewer trained personnel need to be on hand, since intubation can be performed independently by a user. Furthermore, it is advantageous that no additional person is required if the user has to perform a difficult intubation in an emergency situation, in particular unexpectedly. Furthermore, the accuracy and/or quality of intubation can be improved, since the user is assisted by the intubation system. As a result, the user can concentrate more on the patient and, compared to conventional intubation systems, does not have to concentrate as much on the correct handling of the system.

The tube may be any customary tube. In particular, the tube may be an endotracheal tube. In addition, the tube may be a laryngeal tube. The tube may come in different sizes and may be intended for the intubation of patients of all ages. Furthermore, the tube may comprise a cuff in the vicinity of a distal end of the tube. The tube may be made of a plastic, in particular polyvinyl chloride (PVC).

The stylet may be any customary stylet. The stylet may be a flexible and/or a rigid stylet. The stylet may comprise a distal portion which is movable, in particular by means of a movement mechanism. Furthermore, the stylet may have an external diameter which substantially corresponds to an internal diameter of the tube. The stylet may additionally be treatable with a substance which improves the sliding properties between the stylet and the tube. In particular, friction between the stylet and the tube may be able to be reduced.

The laryngoscope may be any customary laryngoscope. In particular, the laryngoscope may be a modular laryngoscope. Modular may mean that the laryngoscope can be assembled and/or that parts of the laryngoscope can in particular be provided in a patient-specific and/or application-specific manner. In general, the laryngoscope may be an instrument which assists the user in inserting the tube. The laryngoscope may comprise at least a handle in addition to the spatula, and the spatula may be designed to be detachably connected to the handle. In particular, the spatula may be able to be connected in a patient-specific and/or application-specific manner. Furthermore, at least parts of the laryngoscope may be intended for single use. For example, the laryngoscope may comprise an imaging module and a main body. The main body may be formed in one piece and/or may be intended for single use. In particular, the laryngoscope may be made at least partially of a plastic—for example, in an injection-molding or additive manufacturing process. The main body may comprise a receiving space, into which the imaging module can be inserted. The laryngoscope may in particular be a video laryngoscope. By means of the video laryngoscope, an anatomical structure may be able to be imaged, thereby generating image data that can be transmitted to a monitor and displayed thereon. The user can view the anatomical structure on the monitor.

The spatula may be a curved spatula or a straight spatula. The spatula may also be a hyperangulated spatula. This may mean that the spatula may have a greater curvature than a conventional, curved spatula. In general, the intubation system according to the invention is not limited to particular spatula geometries. Features of the spatula and/or intubation system described herein may be able to be combined with any spatula. The spatula may be made of a plastic and/or a metal. The spatula may also be able to be disinfected, in particular by wiping, and/or may be able to be autoclaved. For example, the spatula may be able to be made ready for use by cleaning it with a cloth.

In this connection, insertion is to be understood to mean that the tube can be inserted into the patient's trachea. The tube can be guided together with the stylet along the spatula. In particular, the tube may make contact with the spatula in some portions. The tube and/or the stylet may be held at a proximal end by the user during insertion. Furthermore, insertion may also mean the positioning of the tube within the trachea. Positioning may comprise arranging a distal end of the tube relative to an anatomical feature, in particular a feature of the trachea, of the patient. Furthermore, markings may be provided on the tube, which help the user to estimate the position of the distal end of the tube within the trachea. The user can assess the position of the markings relative to the patient's incisors. The aim of insertion is to bring the tube into a specific position in which ventilation can be performed. This position shall, at least substantially, be able to be held.

In particular, the position shall be able to be held while the stylet is being pulled out of the tube. In particular, the tube locking device is provided for this purpose. The tube locking device can prevent the tube from being pulled back relative to the spatula—for example, such that the position can, at least substantially, be held and/or fixed. In this context, substantially able to be held means deviations of, for example, up to 5 cm, in particular up to 2 cm and/or preferably up to 1 cm, from the position. In particular, preventing does not mean the complete absence of any relative movement. Preventing can be understood to mean that, when pulling the stylet back out of the tube, there is a greater relative movement between the stylet and the tube than between the tube and the spatula. It is thus possible to ensure that the tube remains in the trachea when the stylet is pulled back out of the tube. According to the invention, it may be provided that, after the stylet has been pulled back, the tube in the trachea be moved into the position suitable for ventilation. In particular, after pulling the stylet back, the user can set it down and use the now free hand to position the tube.

Furthermore, the stylet may be a video stylet, in particular an endoscope, which comprises an at least substantially rigid shaft, wherein the tube can be pushed onto the shaft. Advantageously, the trachea can thus be seen during insertion of the tube. Furthermore, a video stylet is particularly advantageous in complicated applications, in particular in the case of complicated patient anatomies and/or, for example, when the patient is bleeding heavily. The video stylet may in particular be a bronchoscope. Substantially rigid may mean that the video stylet has such mechanical properties that, during insertion into the trachea, in particular in complicated cases, it is able to deflect laterally by no more than 20%, preferably no more than 10%, and more preferably no more than 5% relative to its longitudinal extent. In other words, the shaft at least substantially retains its shape during insertion and is not bent and/or kinked.

Furthermore, the tube locking device may be modular and may be designed such that it can be optionally attached to the spatula and removed from the spatula. For example, the tube locking device may be a stand-alone product. It may be provided that the user provide and/or attach the tube locking device, depending upon the patient. In particular, any existing intubation system may be provided with the tube locking device. Advantageously, the tube locking device need not necessarily be attached to the spatula. The user can decide whether use of the tube locking device is necessary and/or advantageous.

Furthermore, the spatula and/or the tube may at least partially form the tube locking device. Advantageously, production costs can be reduced. A design can be simple. In particular, it may be provided that the spatula be formed in one piece and include the tube locking device.

Furthermore, the spatula and the tube may jointly form the tube locking device. For example, features of the tube may advantageously interact with features of the spatula. Operational reliability can be improved.

Furthermore, the tube may be able to be released by the tube locking device when necessary. Able to be released may mean that the tube can be made movable, at least substantially, at will, in particular freely, relative to the spatula. In particular, able to be released may mean that the tube can be made movable along its longitudinal axis in both directions along the spatula. Advantageously, the situation can be achieved that, after the stylet has been pulled back, the laryngoscope can be removed from the patient's mouth and/or larynx area, with the tube remaining in the trachea. The operational reliability of the intubation system is thus increased.

Furthermore, the tube locking device may have at least one operating element, by means of which a locked state and/or a released state for the tube can be established. In the locked state, the tube is locked to the spatula in such a way that it is, at least substantially, immobile relative to the spatula. In particular, the tube may be able to be moved jointly with the spatula in the locked state. For example, the user can orient the tube by moving the spatula. Furthermore, the stylet, in the locked state, may be able to be pulled out of the tube. In the released state, the tube may be able to move relative to the spatula. In particular, in the released state, the spatula may be able to be removed from the patient's mouth and/or larynx area, while the tube remains in the patient's trachea. Advantageously, the user can conveniently and/or easily change the states of the tube by operating the operating element. The ease of operation of the intubation system is improved, and the user requires less effort and concentration to operate it. As a result, the quality of a user's handling can increase.

Furthermore, a clamping force can be applied to the tube by operating the operating element. In particular, the clamping force can be applied to the tube in the locked state. Furthermore, the clamping force can be adjusted to a tube and/or a tube-spatula combination. This may mean that it is possible to react to frictional properties of the tube and/or mechanical properties of the tube. By applying the clamping force, a simple design can be achieved, making it possible to provide an operationally reliable and inexpensive intubation system. In particular, any conventional tube can be used with the intubation system. By adjusting the clamping force, it is possible to react to different tubes, thus improving the flexibility and versatility of the intubation system.

Furthermore, the operating element may have a clamping portion which is located opposite the spatula, such that the tube can be clamped between the clamping portion and the spatula. By virtue of the clamping portion, the surface area across which the clamping force acts can be enlarged. The clamping portion may extend across a portion of the tube. In particular, the clamping portion may be designed in such a way that it replicates the shape of the spatula. Advantageously, a homogeneous clamping force can be applied to the tube at least substantially across the entire clamping portion. This may mean that the pressure applied to the tube by the clamping portion is distributed, at least substantially, homogeneously across the clamping portion. For example, the clamping portion may extend along the tube by up to 10 cm, in particular up to 5 cm, preferably up to 2 cm. Furthermore, the clamping portion may, at least substantially, be matched to an external contour of the tube. This may mean that the clamping portion is designed in such a way that it has at least in part a curvature with a radius that at least substantially corresponds to the radius of the tube. Furthermore, the clamping portion and/or the spatula, in particular the region of the spatula against which the tube can be clamped, may comprise a coating which increases a frictional force between the respective portion and the tube. Furthermore, the coating may be deformable such that the corresponding portion, in particular the coating, adapts to the shape of the tube when the tube is clamped. The risk of damaging the tube can be reduced by providing the clamping portion. Furthermore, a greater clamping force can be applied without damaging the tube.

Furthermore, the operating element can be operated by means of an operating force, wherein the operating force is convertible into the clamping force. In particular, this means that the user applies the operating force. Furthermore, it may mean that, for example, an electromechanical actuator is provided, by means of which the operating force can be applied. For example, the user can operate the electromechanical actuator. According to the invention, it is also possible that the electromechanical actuator may be activated automatically. Can be converted may mean that a gear mechanism is provided. Furthermore, a lever arrangement may be provided in order to convert the operating force. The user can adjust the operating force according to the situation and/or according to requirements. Furthermore, the user can obtain information about the tube and/or the locked state by feeling resistance when applying the operating force. For example, if the user notices that an operating force is insufficient to establish a locked state, the user can react accordingly. This increases the operational reliability and efficiency.

Furthermore, the operating element may comprise a lever which is pivotably arranged on the spatula. The lever may be arranged on the spatula in such a way that a clamping force can be applied to the tube by pulling the lever. In particular, the clamping force can be applied by clamping the tube between the operating element and the spatula. The lever may be arranged on the spatula so as to be pivotable about a pivot axle, wherein the pivot axle is at least substantially perpendicular to a main direction of extension of the spatula. The main direction of extension may correspond to the longitudinal direction of the tube during insertion. The user-friendliness is increased by providing the lever. Furthermore, an inexpensive and simple design is thus possible.

In addition, the laryngoscope may comprise the handle on which the spatula is arranged, and the operating element may comprise an actuating portion which is arranged in such close proximity to the handle that the user can actuate the actuating portion while gripping the handle. In particular, this may mean that the user can actuate the actuating portion using the hand used to grip the handle. In particular, the user can actuate the actuating portion using a finger, in particular the little finger or the index finger. Actuation may comprise pulling the operating element. User-friendliness can thus be increased.

Furthermore, the lever may form the clamping portion and the actuating portion. In particular, the lever may be formed in one piece. The lever may be made of a plastic and/or a metal. In particular, the lever may be manufactured in an injection-molding and/or additive manufacturing process. The production costs can thus be kept low. In addition, the lever can be replaced if a different lever is required and/or if the lever is defective. In addition, the lever can be removed in order to clean the intubation system. Assembly may therefore be simple, since the tube locking device may consist of only a few parts.

Furthermore, the tube locking device may be formed on one side. This may mean, for example, that the tube locking device is formed, mounted, and/or arranged on just one, in particular on a single, wall of the spatula. The operation and design can thus be kept simple.

Furthermore, the tube locking device may be designed to act upon the tube from at least two opposite sides of the tube. This may mean that the tube may extend at least partially between the tube locking device, in particular when pulling out the stylet. Functional reliability can thus be increased. Furthermore, usability can be simplified.

Furthermore, the spatula may comprise a lateral tube guide wall, and the tube locking device may be arranged on the tube guide wall. The spatula may for example comprise a main wall. The main wall may extend in the main direction of extension of the spatula and may, at least substantially, define it. A “main direction of extension” of an object is to be understood to mean, in particular, a direction extending parallel to a longest edge of a smallest imaginary cuboid which only just fully encloses the object and which preferably passes through a geometric center and/or a center of mass of the component. The functions of the laryngoscope may be able to be at least substantially provided by means of the main wall. For instance, in the case of a straight laryngoscope, pressure may be able to be applied to the epiglottis by means of the main wall. The main wall may for instance have a length of 5 cm to 15 cm. Some lengths may differ from this. Furthermore, the main wall may extend in a direction which is at least substantially perpendicular to the longitudinal direction of the tube during insertion. The main wall may extend in this direction along, for example, 1 cm to 5 cm, in particular 2 cm to 3 cm. In particular, the extension in this direction may have a length that is greater than the diameter of the tube. Furthermore, the spatula, in particular the main wall, may be flat. This may mean that the main wall extends over an at least substantially shorter length in a third direction. A further wall may extend from the spatula. The further wall may be designed to partially receive an imaging device. In particular, the further wall may define a receiving space for this purpose. Furthermore, a camera and/or a viewing window of a camera may be arranged at a distal end of this wall. The viewing window may comprise a lens arrangement. The wall may also be designed to push the patient's tongue away in a sideways direction. The tube guide wall may for example be part of this wall and/or may be this wall. The tube guide wall may extend along the entire length of the spatula. In other embodiments, the tube guide wall may extend along, for example, up to 70%, in particular 80%, preferably up to 95%, of the length of the spatula. Advantageously, the tube guide wall may be a wall of a conventional spatula. This may mean that the tube locking device can be attached to a wall of a conventional spatula. Advantageously, the tube locking device may be able to be integrated into existing intubation systems through minor design measures. Furthermore, an arrangement on the lateral tube guide wall is advantageous, since it gives a relatively large degree of freedom of movement in the lateral direction compared to the other directions. A movement of the tube in the lateral direction may therefore be advantageous.

Furthermore, the tube locking device may comprise at least one rotatable locking element. In general, the locking element may be at least partially inset into a wall of the spatula. Rotatable may in particular also include pivotable. The locking element may be rotationally symmetrical. In other embodiments, the locking element is not rotationally symmetrical. In particular, the locking element may be mounted eccentrically. In other embodiments, the locking element may also be rotatable about, for example, two or three axes. The locking element may be intended to interact with the tube. To this end, the locking element may comprise a coating—for example, in order to increase friction. Rotatable locking elements have the advantage that they require a comparatively small installation space. Furthermore, constant rubbing of the tube against the locking element, as may occur with a non-rotatable locking element, can at least largely be avoided. Ease of use can be increased, and the risk of damage to the tube can be reduced.

Furthermore, the locking element may have a locking direction of rotation and a release direction of rotation which is opposite to the locking direction of rotation. In the locking direction of rotation, rotation of the locking element may at least only be possible by applying a relatively large force. In particular, rotation in the locking direction of rotation may not be possible. Furthermore, in some embodiments, the locking element may be rotatable in the locking direction of rotation until a locking action takes place—for example, through an angle of at most 90 degrees, at most 60 degrees, at most 45 degrees, or at most 30 degrees. The position at which the locking action takes place may be a locked position. In the locking direction of rotation, the locking element may, at least substantially, prevent the movement of the tube relative to the spatula. Advantageously, incorrect operation can at least largely be avoided. This means that the operational reliability of the intubation system can be increased.

Furthermore, the locking element may be rotatable about a rotation axle which, in a pushed-forward state of the tube, is arranged, at least substantially, perpendicularly to a longitudinal axis of the tube in a region of the locking element. A simple and space-saving design can thus be achieved.

In addition, the rotatable locking element may comprise a clamp which is designed to prevent the tube, by clamping it, from being pulled back relative to the spatula. The clamp may comprise a clamping portion which in particular is intended to interact with the tube. The clamp may comprise a coating and/or profiling to increase a clamping force, wherein the clamping force is a force that opposes movement of the tube out of the trachea. By way of example, the clamp may also comprise a serrated clamping portion. In particular, the clamp may in particular be able to be activated passively by pulling the tube back. This may mean that the clamp can be activated by the user pulling back the stylet between the clamp and the spatula. In this connection, clamping may also mean blocking. This may mean that the locking element is designed to reduce a distance between the clamp and the spatula by pulling the tube back. For example, the mode of operation of the clamp may at least substantially correspond to the mode of operation of a rope clamp. Advantageously, a tube locking device can be provided through relatively simple design measures. This can be characterized in particular by a small space requirement.

In addition, the rotatable locking element may comprise a guide roller for the tube. The tube locking device may also comprise a plurality of guide rollers—for example, two, three, or four. The guide roller may have, along its axis of rotation, a width that is at least equal to the diameter of the tube. In some embodiments, the guide roller may be wider than the diameter of the tube. Furthermore, the guide roller may comprise a recess perpendicular to the axis of rotation along the entire circumference. The recess may have a curvature with a radius that at least substantially corresponds to the radius of the tube. In particular, the guide roller may have a U-shaped and/or concave profile. Furthermore, the guide roller may comprise two lateral guide walls which are designed to guide the tube along the longitudinal axis of the tube. By providing at least one guide roller, a cost-effective intubation system can be provided. Furthermore, the guide roller can be arranged in a space-saving manner. In particular, a guide roller may be able to be provided inset into the spatula relatively easily.

Furthermore, the tube locking device may comprise a tensioning element which is arranged on the locking element and pre-tensions the locking element. The tensioning element may be designed to store potential energy and release it to prevent the tube from being pulled back along the spatula. The tensioning element may additionally be able to be deformed by pulling the tube back along the spatula. A tensioning force can increase as a result of the deformation. In general, a clamping force can be increased by the tensioning element. In particular, a greater clamping force can also be achieved in the same amount of installation space.

Furthermore, the tensioning element may be a leg spring. Leg springs can be characterized in particular by a small required installation space, can be cost-effective, and can be provided in a simple design.

Furthermore, the tube locking device may comprise at least one projection which is designed to prevent the tube from being pulled back, by engaging behind at least part of the tube. In particular, the projection may be arranged on the spatula. Furthermore, the projection may be a distal end of the wall which comprises the camera of the video laryngoscope. The tube may comprise a portion which is designed to interact with the projection. The projection may also be understood as a mechanical stop. The tube can be pressed laterally against the mechanical stop by the user in order to prevent it from being pulled back. Using the projection and/or the mechanical stop to prevent pulling back is a simple and cost-effective method for preventing pulling back. Furthermore, moving parts can be avoided. Furthermore, such an intubation system may be able to be cleaned, disinfected, and/or autoclaved easily.

In addition, the tube locking device may comprise a keeper element which is designed to prevent the tube from being pulled back, by engaging behind at least part of the tube. The keeper element may be provided in addition to the projection. For example, the keeper element may comprise a further projection. In addition, the keeper element may comprise a lug, in particular a plastic lug. Furthermore, the keeper element may comprise a pin, a bolt, and/or the like. The keeper element may be able to be mounted on the spatula. Furthermore, the keeper element may be formed by the spatula. The keeper element can improve the effect of the locking device. The likelihood of a malfunction can be reduced. Furthermore, this can be achieved through simple design measures in a small installation space.

Furthermore, the keeper element may be arranged at a distance from the projection, and the distance may at least substantially correspond to an external diameter of the tube. The distance to the projection means, in particular, the smallest distance to the projection. The tube may extend between the projection and the keeper element, in particular along the longitudinal axis of the tube. The tube may make contact with the projection and the keeper element on opposite sides. For example, the projection may comprise a step. The upper part of the step may be arranged proximally to the tube, and the lower part of the step may be arranged distally to the tube. For example, the distance may be up to 3 cm, in particular up to 2 cm, preferably up to 1.5 cm. For example, frequently used spatulas can be paired with frequently used tubes. This means that a spatula of a standard size can be provided with the keeper element at a distance from the projection which at least substantially corresponds to the external diameter of a tube that is commonly used with the spatula. Multiple tube sizes can be provided for one spatula. The keeper element may be designed to make the distance adjustable. For example, the keeper element may be eccentrically rotatable, wherein the distance can be varied by rotating the keeper element. Advantageously, the tube can be guided by such an arrangement. In particular, the tube may be guided along the longitudinal axis. This results in a lower risk of malfunction and greater operational reliability. The tube is easier to keep in a position in which part of the tube engages behind the projection.

Furthermore, the tube may comprise a circumferential widening, and at least part of the tube locking device can engage behind the circumferential widening. The circumferential widening may be formed by the tube and/or attached to the tube. In particular, the widening can be attached to the tube. For example, the widening may comprise a ring, an attachment, a projection, a bulge, and/or the like. The widening may extend around the entire circumference. In other embodiments, the widening extends around part of the circumference. The widening may be displaceable in the longitudinal direction of the tube, and/or a position may be variable in the longitudinal direction of the tube. In particular, the tube can be adapted in a patient-specific, spatula-specific, and/or application-specific manner. The flexibility and versatility of the tube and/or the intubation system can be improved.

Furthermore, the intubation system may comprise at least one latching element, in particular a latching tongue, which, when force is applied in a first direction, can be moved into a released position in which the latching element allows the stylet, together with the tube which is pushed onto the stylet, to be pushed forward relative to the spatula, and which, when force is applied in a second direction, can be moved into a locked position in which the latching element prevents the tube from being pulled back relative to the spatula. This may mean that the tube locking device is designed in the manner of a cable tie. The tube may be able to be moved in one direction along the longitudinal axis, and, in the locked position, pulling back can be prevented in the opposite direction. The tube may be able to be moved in the opposite direction into the locked position. This may mean that the tube is movable beyond the locked position in the first direction. If the tube is moved in the opposite direction, the locked position can be reached again, and further pulling back can be prevented. In the locked position, the tube may be able to be moved jointly with the spatula and/or may, at least substantially, be immovably fixed to the spatula. In particular, the latching element may be movable in a direction radial to the longitudinal direction of the tube. The latching element may extend at least in part at an angle away from the tube in an unloaded state. The latching element can be placed against the tube by applying a load in a radial direction. Particularly in an angled state, the latching element may be designed to prevent pulling back. Furthermore, the latching element may be designed to prevent pulling back up to a limit pull-back force, wherein the limit pull-back force is a force that brings about the pulling-back of the tube. Above the limit pull-back force, pulling back may be possible. This may lead to greater safety, since, in case of emergency, the user can apply a force that exceeds the limit pull-back force. Furthermore, by providing the limit pull-back force, it is possible to provide for the removal of the laryngoscope from the mouth and/or larynx area. In general, by providing the latching element, an effective and efficient intubation system can be provided.

Furthermore, the tube locking device may comprise a plurality of latching projections, in particular latching teeth, which each define a locked position and which are arranged one behind the other along a longitudinal axis of the tube, and the latching element may, by engaging behind at least one of the latching projections, be designed to prevent the tube from being pulled back. Some features may be the same as some features in connection with the latching element. In particular, the latching projections may be formed in a manner similar to the latching tongues. Furthermore, the latching projections may be movable along the longitudinal axis of the tube. The latching projections may in particular be movable in one direction and may be less movable, in particular at least largely immovable, in a direction opposite to said direction. Furthermore, for example, a stop and/or a wall may be provided, which limits a mobility of the latching projections particularly in one direction. Such a tube locking device may be designed in the manner of a cable tie. By providing multiple locked positions, the versatility of the intubation system can be improved. It is possible to easily react to patient anatomies and/or to situations during use.

Furthermore, the tube locking device may comprise a retaining ring which is attached to the spatula, wherein the stylet, together with the tube which is pushed onto the stylet, can be pushed forward through the retaining ring relative to the spatula. For example, the retaining ring may also be understood to mean a retaining tunnel and/or retaining channel. In particular, it can be understood to mean a device through which the tube can be passed and/or which can at least largely encompass the entire circumference of the tube. For example, the retaining ring may extend up to 10 cm, in particular up to 5 cm and/or preferably up to 2 cm, along the longitudinal direction of the tube. A high degree of operational reliability and a reliable intubation device can thus be provided.

The tube locking device may also be designed to prevent the tube from being pulled back, by applying a frictional force to the tube. For example, the frictional force may be brought about by bringing the spatula into contact with the tube. The tube may be designed to make contact at least in part. By way of example, the tube may to this end comprise a frictional force-inducing attachment, wherein the frictional force-inducing attachment may be slipped and/or pushed onto the tube. Furthermore, the pulling-back of the tube may be able to be brought about by adhesive interaction between the tube and the spatula. Such a tube locking device may be designed in a manner similar to a tube locking device which is designed to prevent pulling back by means of a frictional force. Both offer the advantage of a simple design. Furthermore, in both cases, it is possible to avoid the situation where, in particular, sharp edges must be provided. Sharp edges can cause injury to the patient.

Furthermore, the tube locking device may comprise at least one rubberized, profiled, and/or rough surface. The frictional force can thus be brought about. Such a surface may be provided on at least part of the tube and/or on at least part of the spatula. Furthermore, a surface pairing on the tube and spatula may be provided, which is designed to increase the frictional force. The surface may be made of a metal, a plastic, and/or a composite material. In particular, the surface may be made of rubber and/or a rubber-like material. A band, a sheath, and/or the like comprising the surface may be able to be attached to the tube. For example, the band, the sheath, and/or the like may extend up to 10 cm, in particular up to 5 cm, preferably up to 2 cm, along the longitudinal direction of the tube. In particular, the tube locking device may comprise a rubber band which can be arranged on the tube.

Such a design measure can be integrated into conventional intubation systems. Furthermore, it is cost-effective and can be implemented in a very small installation space.

Furthermore, the tube locking device may comprise at least one bracket, into which the tube can be hooked. The bracket may be designed to clamp the tube on one side, two sides, and/or multiple sides. In particular, the bracket may be designed to apply a clamping force to the tube. The tube may be subjected to a clamping force in such a way that the tube can be held between the spatula and the bracket. In particular, the tube may be able to be clamped against the spatula. Furthermore, the tube may be able to be clamped between two bracket elements. The bracket, in particular the bracket elements, may at least partially clamp around the tube. The bracket and the spatula and/or the bracket elements may be spaced apart from each other by a bracket gap when the tube is not hooked in. An opening can thus be defined. The bracket gap and/or the opening may be, at least substantially, smaller than the external diameter of the tube. The bracket may be movable and/or elastically deformable at least in part. The bracket gap and/or the opening for hooking the tube in can thus be enlarged. In particular, the bracket may be designed in the manner of a hose clip and/or a hose bracket. The bracket may comprise a coating on one surface, in particular on the inner side, wherein the surface faces toward the tube in a hooked-in state. In particular, able to be hooked in may be understood to mean that the tube can be hooked in as required and/or selectively. For example, the user can hook in the tube. In particular, the user can hook in the tube after the tube has been inserted into the trachea, for example. By virtue of the bracket, a high degree of reliability of the tube locking device can be achieved.

In addition, the tube can be hooked sideways into the bracket and can be unhooked sideways from the bracket. Sideways may mean, in particular, at least substantially perpendicular to the longitudinal direction of the tube and/or to the main direction of extension of the spatula. The opening may open sideways relative to the longitudinal direction of the tube. There is a greater freedom of movement in the sideways direction in the patient's oral cavity. The risk of injury to the patient when hooking and/or unhooking can thus be reduced.

The tube locking device may also comprise a clamping spring which is designed to prevent the tube, by clamping it, from being pulled back. Furthermore, the tube locking device may comprise a clamping element which is pressed against the tube by the clamping spring in order to clamp the tube. Furthermore, the clamping spring itself may press at least in part against the tube in order to clamp the tube. The clamping spring may comprise a coil spring, a ring spring, a disk spring, and/or the like. Advantageously, a cost-effective intubation system can thus be provided.

Furthermore, the spatula may comprise a lateral tube guide wall, and the clamping spring may be designed to press the tube, in order to clamp it, against the tube guide wall by applying a lateral force. There is a greater freedom of movement in the sideways direction in the patient's oral cavity. The risk of injury to the patient when hooking and/or unhooking can thus be reduced. Furthermore, the space required for the tube locking device can be reduced.

In particular, the clamping spring may be a leaf spring. The tube can thus be easily clamped across a relatively large contact area between the clamping spring and the tube. The leaf spring may be able to be attached to the spatula by at least two fastening elements. For example, the fastening elements may comprise a lug, in particular a plastic lug, a pin, and/or a bolt. Furthermore, the fastening elements may be formed by the spatula.

Furthermore, the stylet can be pulled back out of the tube by a single user using two hands. In particular, this can reduce the financial and/or time-related costs of using an intubation system, since no additional user needs to be available. Furthermore, the efficiency of the intubation system can be increased. In addition, the quality of use of an intubation system can be increased, since coordination between at least two users can increase complexity. Furthermore, it is possible to react spontaneously and/or flexibly to complicated situations. In particular, this can be done even if no additional, in particular trained, user is present.

The present invention will be described by way of example below with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using number words, such as first, second, third object, etc., these are used to name and/or assign objects. Accordingly, for example, a first object and a third object may be included, but not a second object. However, a number and/or sequence of objects could also be derived using number words.

In the drawings:

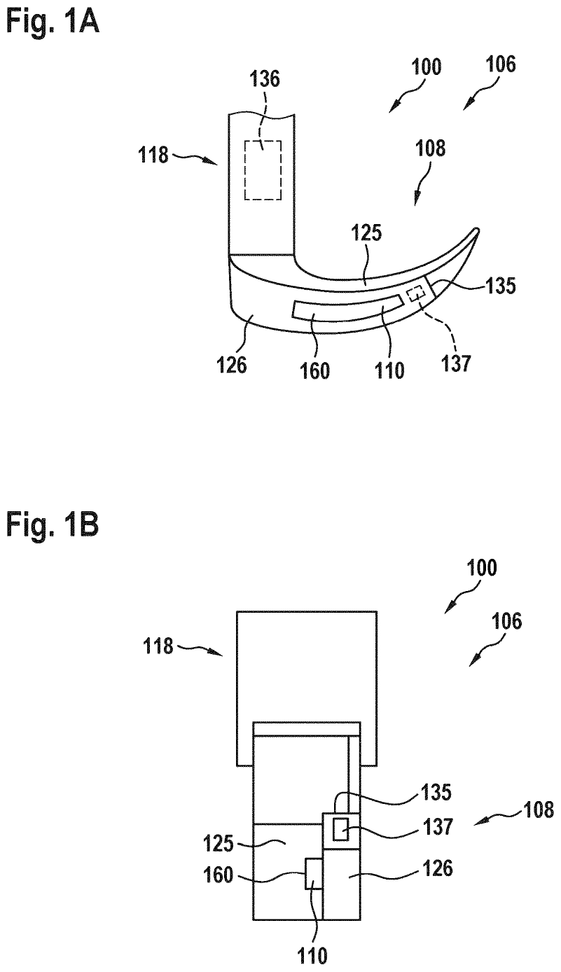

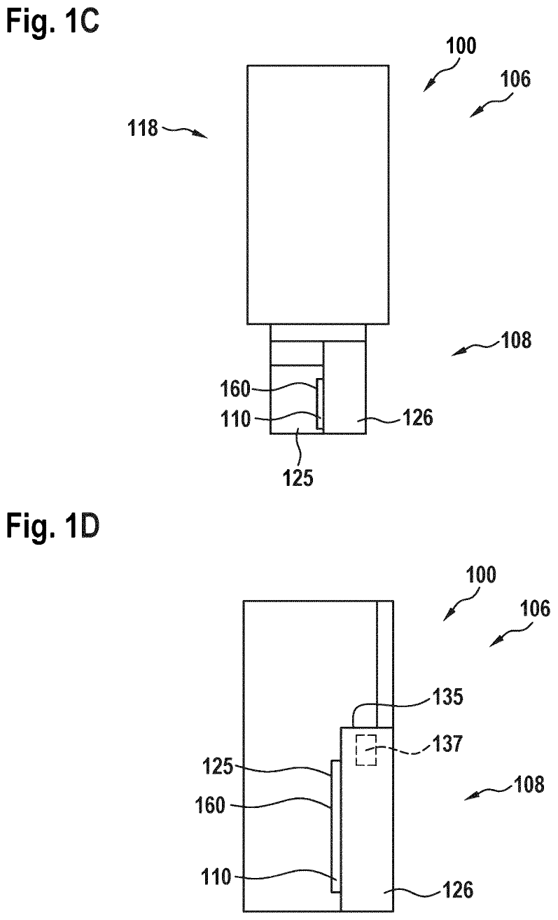

FIG. 1A shows a laryngoscope in a schematic side view;

FIG. 1B shows the laryngoscope in a schematic view from the front;

FIG. 1C shows the laryngoscope in a schematic view from behind;

FIG. 1D shows the laryngoscope in a schematic view from below;

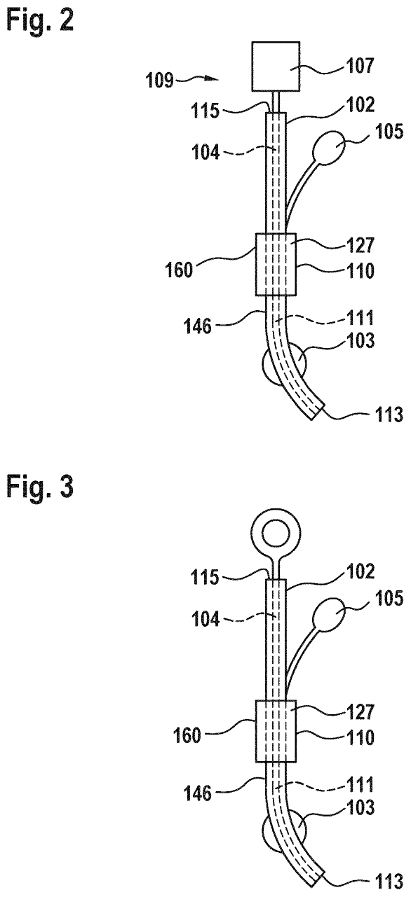

FIG. 2 shows a schematic view of a tube and a video stylet;

FIG. 3 shows a schematic view of the tube and a stylet;

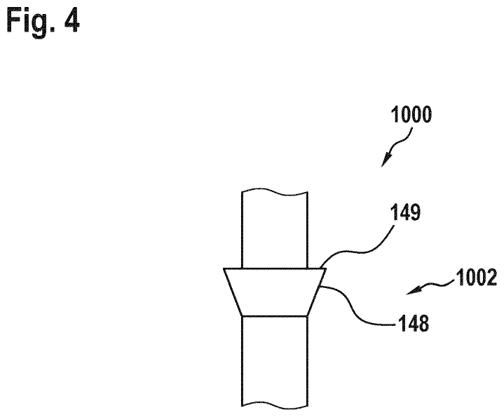

FIG. 4 shows a schematic view of a portion of a tube;

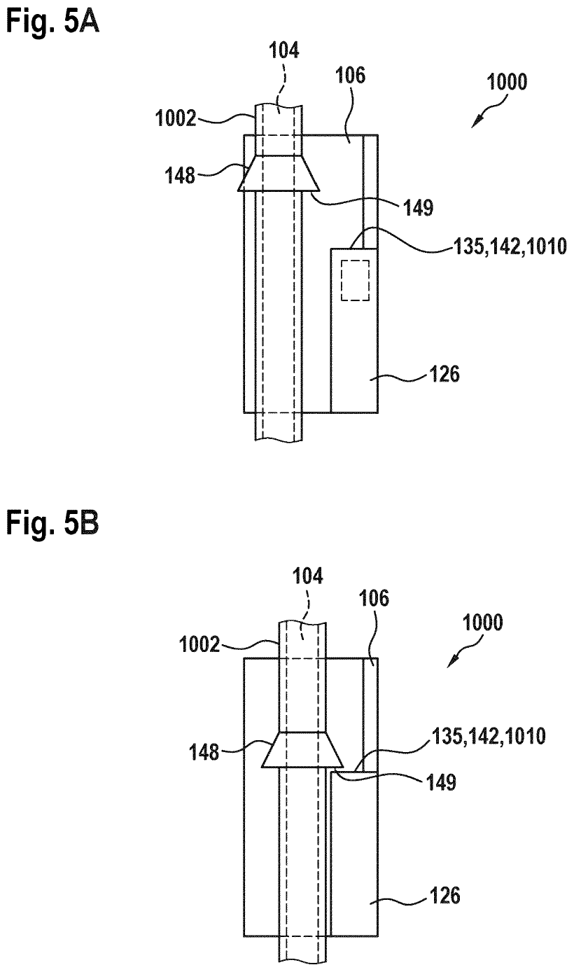

FIG. 5A shows a schematic view of a laryngoscope and the tube from below;

FIG. 5B shows a schematic view of the laryngoscope and the tube from below;

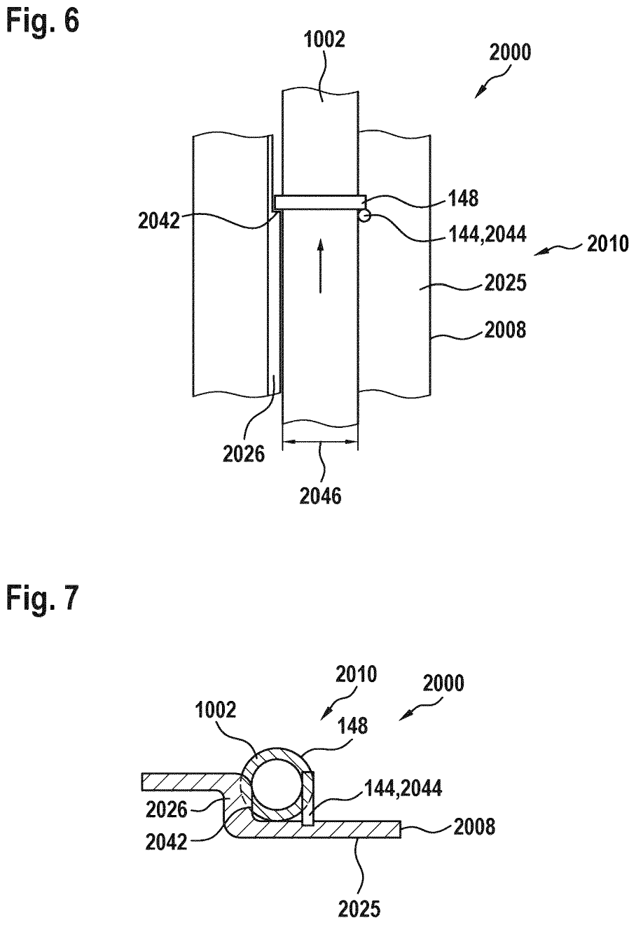

FIG. 6 shows a schematic view of another embodiment of an intubation system from below;

FIG. 7 shows a sectional view of the intubation system from a proximal side;

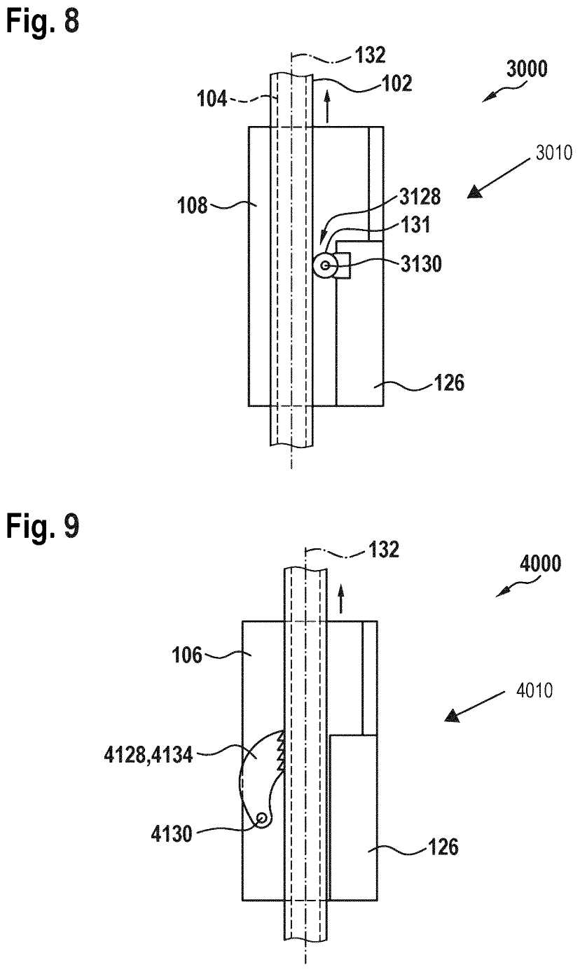

FIG. 8 shows a schematic view of an intubation system from below;

FIG. 9 shows a schematic view of an intubation system from below;

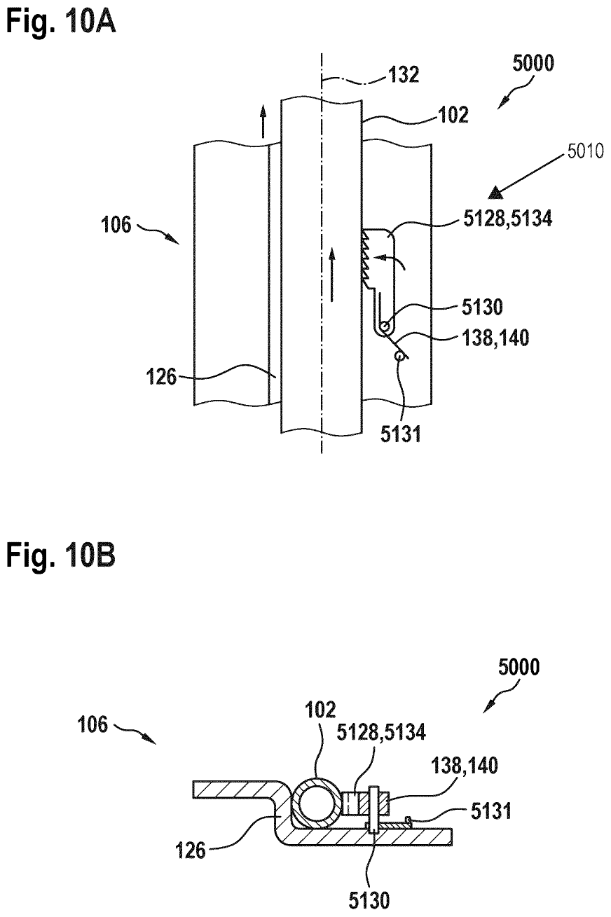

FIG. 10A shows a schematic view of another embodiment of an intubation system from below;

FIG. 10B shows a sectional view of the intubation system from a proximal side;

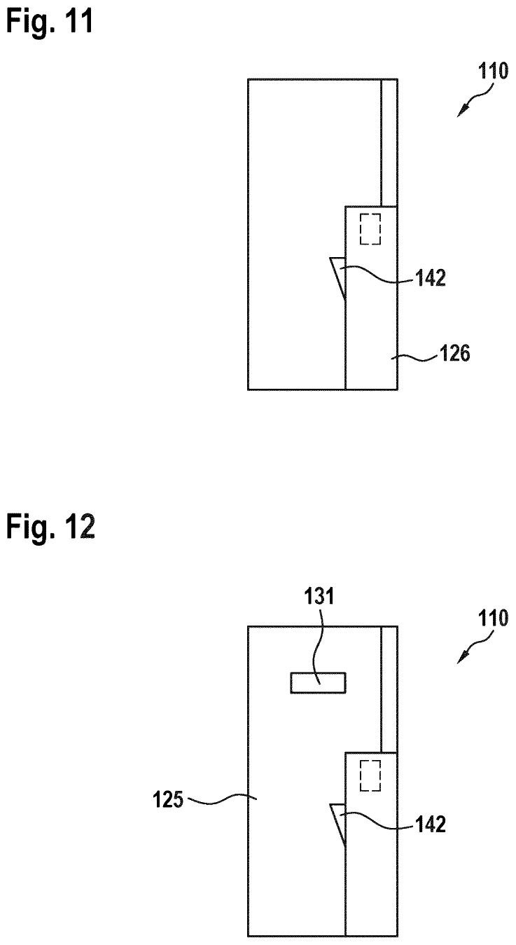

FIG. 11 shows a schematic view of an intubation system from below;

FIG. 12 shows a schematic view of an intubation system from below;

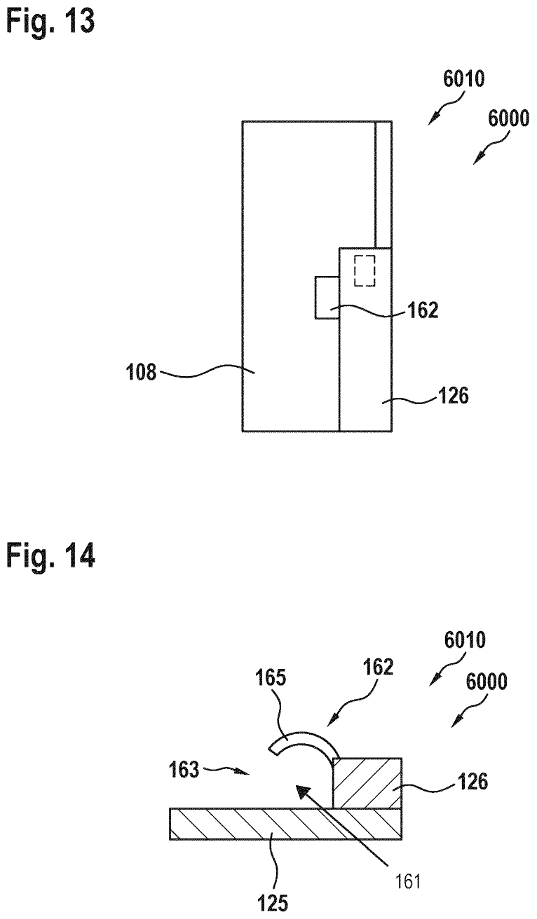

FIG. 13 shows a schematic view of another embodiment of an intubation system from below;

FIG. 14 shows a sectional view of the intubation system from a proximal side;

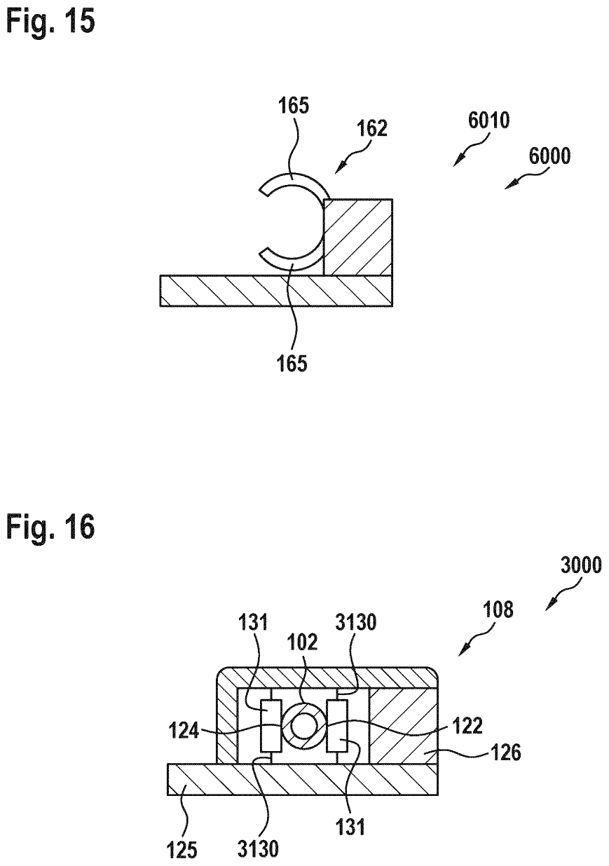

FIG. 15 shows an alternative embodiment of the tube locking device;

FIG. 16 shows a development of the intubation system in a sectional view from a distal side;

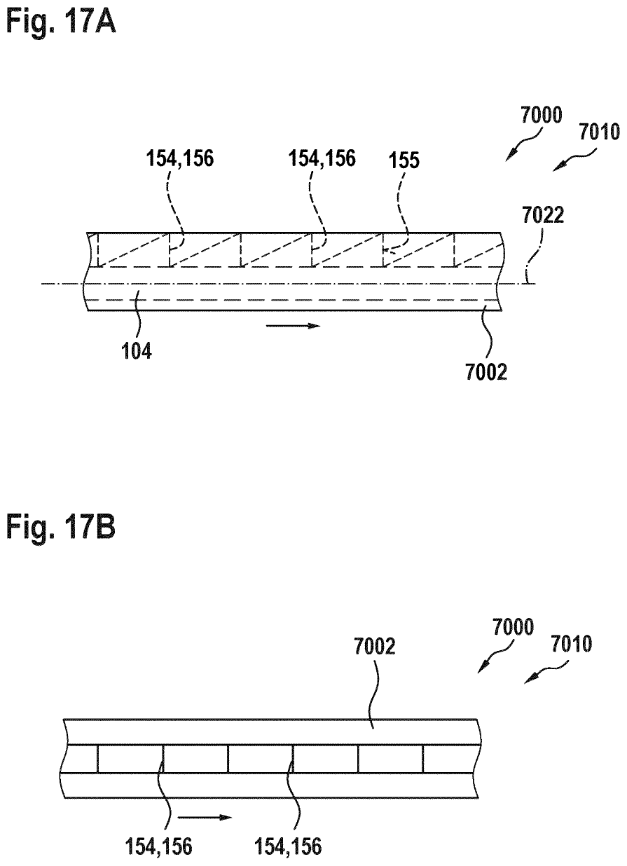

FIG. 17A shows a tube of another embodiment of an intubation system;

FIG. 17B shows the tube in another view;

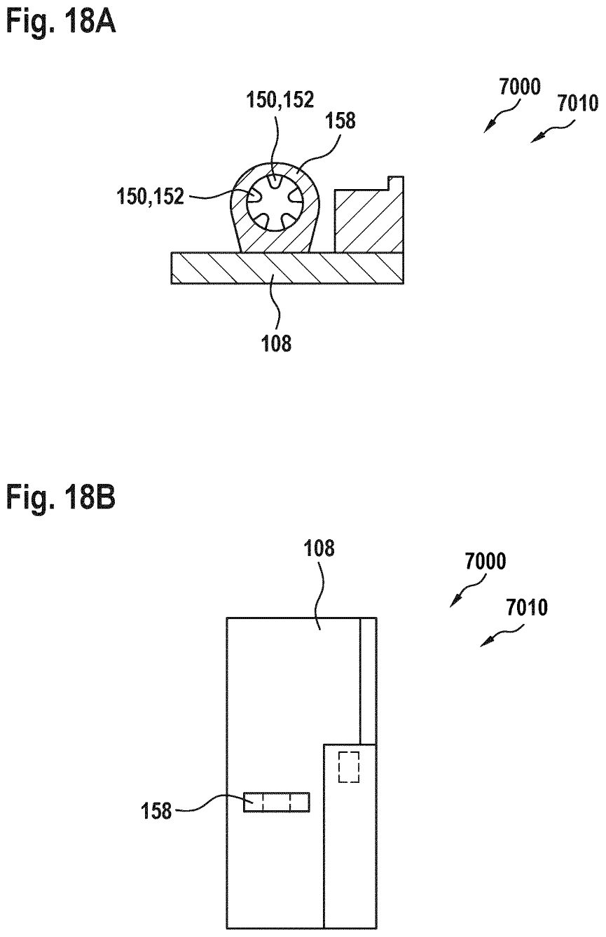

FIG. 18A shows a sectional view of the intubation system;

FIG. 18B shows a schematic view of the intubation system from below;

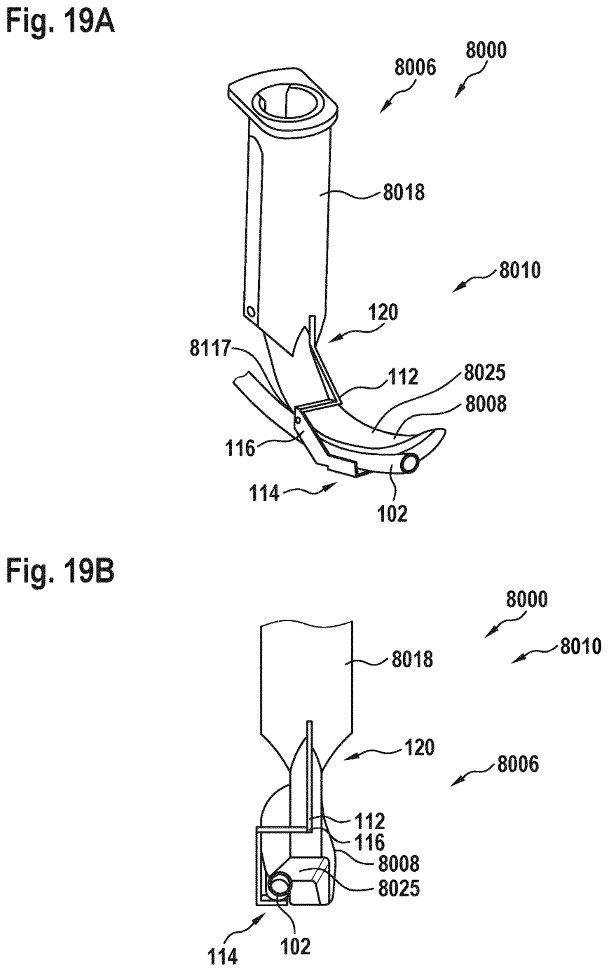

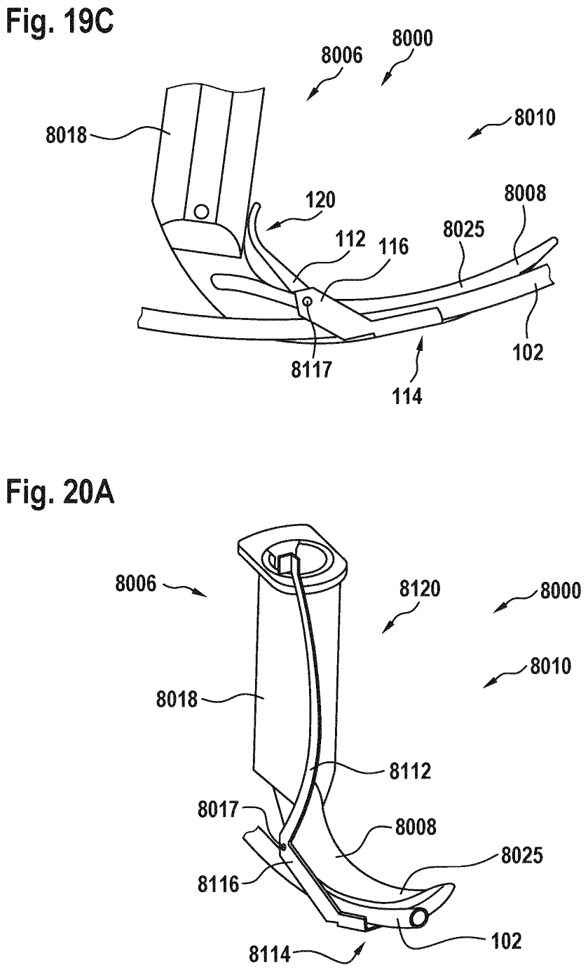

FIG. 19A shows a perspectival schematic view of an intubation system;

FIG. 19B shows a schematic frontal view of the intubation system;

FIG. 19C shows a schematic side view of the intubation system;

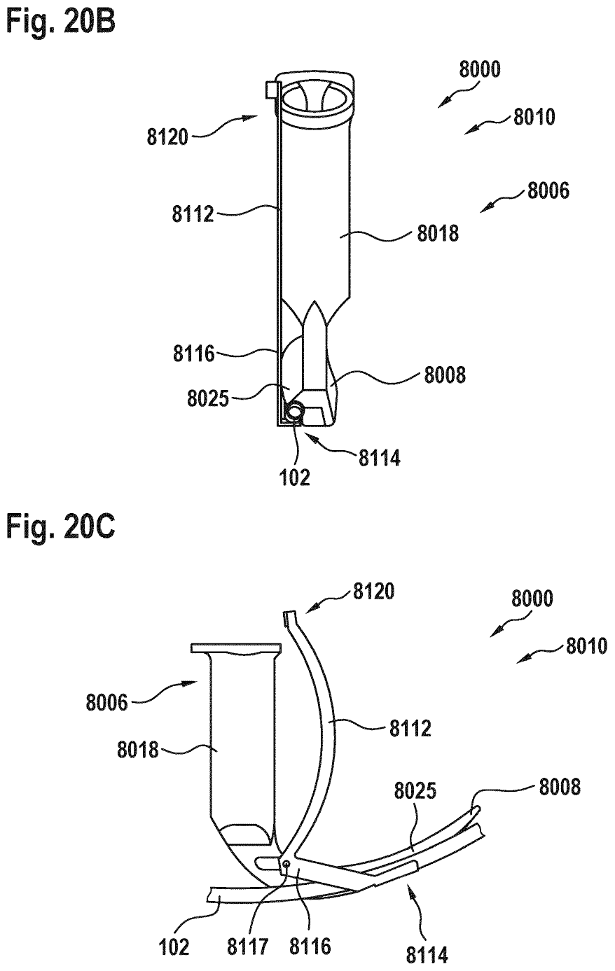

FIG. 20A shows a perspectival schematic view of an intubation system;

FIG. 20B shows a schematic frontal view of the intubation system;

FIG. 20C shows a schematic side view of the intubation system;

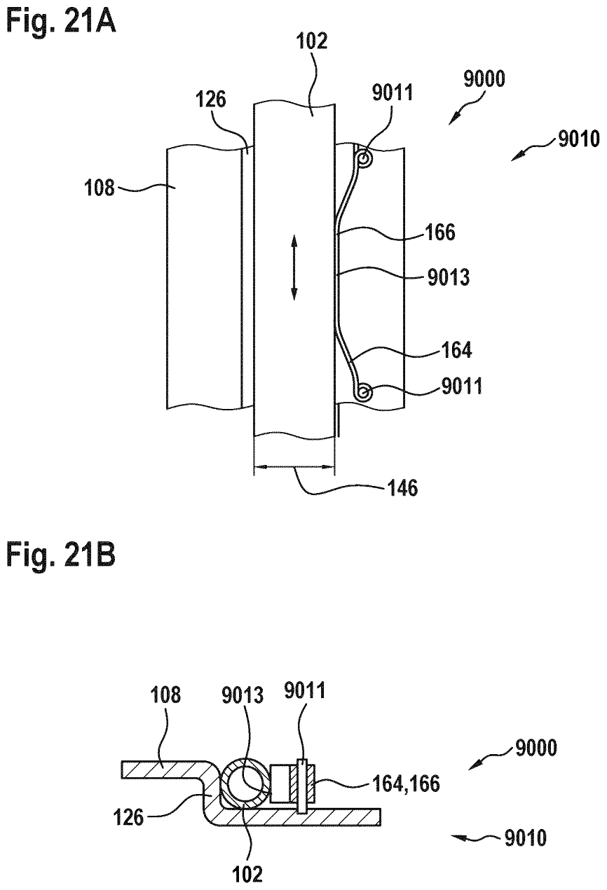

FIG. 21A shows a schematic view of another embodiment of an intubation system from below; and

FIG. 21B shows a sectional view from a distal side of the intubation system.

FIGS. 1A to 1D show different schematic views of a laryngoscope 106, which is part of an intubation system 100. The laryngoscope 106 is shown in a side view in FIG. 1A, in a view from the front in FIG. 1B, in a view from behind in FIG. 1C, and in a view from below in FIG. 1D. The features described below are not to be understood as limiting. There are various types of laryngoscope that can be combined with the features according to the invention. In this connection, the laryngoscope 106 of FIG. 1 is to be understood as an example.

The laryngoscope 106 comprises a handle 118 and a spatula 108. The spatula 108 is made of metal and has a curved shape which enables a user to see and manipulate a patient's larynx. The spatula 108 extends out from the handle 118 at least substantially in a main direction of extension. In the main direction of extension, the spatula 108 curves toward the handle 118 such that the laryngoscope 106 is crescent-shaped. The spatula 108 comprises a main wall 125 and a lateral tube guide wall 126. The main wall 125 is provided for indirectly manipulating the patient's epiglottis. The spatula 108, in particular the main wall 125, is guided in front of the patient's epiglottis, and the laryngoscope 106 is pulled by the user in the direction of the handle 118 of the laryngoscope 106.

The curved spatula 108, in particular the main wall 125, is flat and wide and curves with a slight curvature. The length and width of the spatula 108 are application-specific and manufacturer-specific. In general, the spatula 108 is long enough to reach the patient's larynx, while being narrow enough to allow a clear view of the surrounding area, in particular the patient's vocal cords. For example, the schematically shown spatula 108 has a length of 98 mm and has a width of 14 mm at the distal end. The lateral tube guide wall 126 extends along the main wall 125 and tapers along the main direction of extension, converging, at least substantially, to a point at the distal end of the main wall 125. Furthermore, it can be seen that the lateral tube guide wall 126 comprises a step 135 in a distal end region of the spatula 108. The step 135 can also be referred to as a projection 142. Distally from the step, the lateral tube guide wall 126 is narrow, in particular narrower than the rest of the lateral tube guide wall 126.

The surface of the spatula 108 is smooth in order to enable easy cleaning and/or sterilization. The spatula 108 can be detachably connected to the handle 118 by way of a fastening mechanism, which is not shown in detail.

The laryngoscope 106 comprises an imaging device 136; the laryngoscope 106 is therefore a video laryngoscope. The imaging device 136 is at least partially received by the handle 118. Furthermore, the imaging device 136 extends at least partially along the lateral tube guide wall 126. The imaging device 136 comprises a camera 137 which is arranged in a distal end region of the spatula 108, in particular of the lateral tube guide wall 126. An anatomical structure, in particular the larynx, of the patient can be imaged by means of the imaging device 136 and/or the camera 137. The camera 137 is in particular arranged in the region of the step 135 and/or the projection 142. The step 135 and/or the projection 142 defines a surface which points toward the larynx when the spatula 108 is inserted into the patient's oral cavity.

The intubation system 100 additionally comprises a tube 102 and a stylet 104, which are shown schematically by way of example in FIGS. 2 and 3. In both examples shown, the tube 102 is an endotracheal tube. However, the present invention is not limited to endotracheal tubes. In FIG. 2, the tube 102 is shown schematically. The tube 102 is designed to be inserted into the patient's trachea and to provide an unimpeded flow of air into the lungs, even in cases of impaired breathing or unconsciousness. The tube 102 is tubular in shape and is made of polyvinyl chloride (PVC). In principle, the tube is available in different sizes to meet the individual needs of the patient. At a distal end 113 of the tube 102, the tube 102 comprises an opening, through which air can be introduced into the lungs, and, at a proximal end 115, the tube 102 is designed to be connected to a ventilation device. Furthermore, the tube comprises a cuff 103 in the vicinity of the distal end 113, and a balloon 105 which is designed to fill the cuff 103 with air when the tube 102 is inserted into the patient's trachea. Prior to insertion of the tube 102, the cuff 103 is at least substantially flat, so that an external diameter 146 of the tube 102 remains at least substantially unchanged.

The tube 102 is at least partially pushed onto a stylet 104, in particular a video stylet 109. Alternatively, it is shown in FIG. 3 that the tube can be pushed onto a stylet 104 that is not a video stylet. FIG. 3 shows the same tube 102 used with a stylet 104 that differs in a few features. In both cases, the stylet 104 comprises a rigid shaft 111. The stylet 104 and the video stylet 109, in particular the shaft 111, has an external diameter which substantially corresponds to an internal diameter of the tube 102. The shaft 111 gives the tube 102 a flexural rigidity that facilitates insertion of the tube 102 into the trachea. The shaft 111 extends along the entire tube 102. Compared to the stylet 104 of FIG. 3, the video stylet 109 of FIG. 2 has the advantage that, in particular, the interior of the trachea can be imaged in an inserted state. Other types of stylet can also be provided, and the two stylets 104, 109 that are shown are merely examples.

To insert the tube 102, it is pushed through the patient's mouth into the trachea while pushed onto the stylet 104, 109. The laryngoscope 106 is used to make the opening of the trachea visible, so that the tube 102 can be positioned accurately. The stylet 104, 109, together with the tube 102 which is pushed onto the stylet 104, 109, is pushed forward relative to the spatula 108. After insertion into the trachea, the stylet 104, 109 is pulled out of the tube to allow ventilation. The balloon 105 is used to inflate the cuff 103 in order to keep the tube 102 in position in the trachea, for example.

Furthermore, the laryngoscope 106, in particular the spatula 108 and/or the lateral tube guide wall 126, and the tube 102 comprise a tube locking device 110. The tube locking device 110 is designed to prevent the tube 102 from being pulled back relative to the spatula 108 when the stylet 104, 109 is pulled back out of the tube 102 after having been pushed forward, in particular into the trachea. In particular, a single user can pull the stylet 104 back out of the tube 102 using two hands. The user can grip the laryngoscope 106 with one hand and can use the other hand to pull the stylet 104, 109 back out of the tube. The user need not hold the tube 102 throughout. By virtue of the tube locking device 110, the tube 102 is prevented from moving relative to the spatula 108, and in particular the tube 102 remains in the trachea. In the case shown in FIGS. 1A to 1D, FIG. 2, and FIG. 3, the spatula 108 and the tube 102 jointly form the tube locking device 110. In this connection, the spatula 108 and/or the tube 102 at least partially forms the tube locking device 110. However, the tube locking device 110 shown in these figures may also be formed by only the tube or only the spatula. In principle, features of the tube locking device 110 may be identical.

The spatula-side tube locking device 110 may comprise a rubberized, profiled, and/or rough surface 160. As shown in FIGS. 1A to 1D, the tube locking device 110 comprises a rubberized surface 160. The tube locking device 110 extends along approximately 50% of the length of the spatula 108 in a middle portion of the spatula 108. Furthermore, it can be seen that the tube locking device 110 is formed on one side. This means that the tube locking device 110 interacts with the tube 102 only from one side of the spatula 108 and/or that only one side of the tube 102 is acted upon by the tube locking device 110. In particular, it can be seen that the tube locking device 110 is arranged on the lateral tube guide wall 126 and extends along 50% of the length of the tube guide wall 126, in particular in the main direction of extension of the spatula 108. The tube 102 is guided along the main direction of extension of the spatula 108. Therefore, the tube locking device 110 extends along the guide direction of the tube 102 during insertion of the tube 102 into the patient's trachea. In the case shown, the tube locking device 110, in particular the surface 160, is permanently provided on the spatula 108. In other cases, the tube locking device 110, in particular the surface 160, may be modular and may be designed such that it can be optionally attached to the spatula 108 and removed from the spatula 108. For example, the tube locking device 110 may be able to be glued on and/or clipped on. By means of friction, the surface 160 prevents the tube 102 from being pulled back when the stylet 104, 109 is pulled back relative to the spatula 108.

As an alternative or in addition, the tube locking device 110 is and/or can be arranged on the tube. In the case shown, the tube locking device 110 is additionally arranged on the tube 102. However, the tube locking device 110 may also be arranged solely on the tube. In FIG. 2 and FIG. 3, the tube locking device 110 is shown as a band 127 which is pushed onto the tube 102. The band 127 comprises a rubberized surface 160 on its outer side. In particular, the entire band 127 is made of rubber. The band 127 can be pushed onto the tube 102 by the user if the user wishes to improve the function of the tube locking device 110. For example, it may be necessary to provide the band 127 if the patient is bleeding and/or intubation is difficult. However, it may also be provided that the tube 102 together with the band 172 pushed thereon be made available to the user, in particular in sterile packaging. In the case shown, the band 127 has a length of 30 mm and is arranged in a region proximal to the cuff 103. The region extends from a middle region of the tube 102 into a distal region of the tube 102. The tube locking device 110 is designed to prevent the tube 102 from being pulled back, by applying a frictional force to the tube 102. For example, in order to prevent the tube 102 from being pulled back relative to the spatula 108, the user presses the tube 102, in particular the band 127, against the rubberized surface 160 of the spatula 108 while pulling the stylet 104, 109 back out of the tube 102. The tube 102 can be released by the tube locking device when necessary, in particular by the user varying the applied pressure of the tube 102 against the spatula 108, and thereby the frictional force.

FIGS. 4, 5A, and 5B show another embodiment of a tube locking device 1010. This may be provided on the laryngoscope 106 and/or a tube 1002 as an alternative or in addition to the tube locking device 110. FIG. 4 schematically shows a portion of the tube 1002 with a tube-side part of the tube locking device 1010. FIGS. 5A and 5B show the tube 1002 and the laryngoscope 106 in a schematic view from below, looking toward the laryngoscope 106.

The tube 1002 is at least substantially similar to the tube 102, and for this reason, with regard to the features thereof, reference can be made to what has been stated above. The difference is that the tube 1002 comprises a circumferential widening 148, behind which at least part of the in particular spatula-side tube locking device 1010 can engage. The circumferential widening 148 extends along the longitudinal direction of the tube 1002 around the entire circumference, is designed as a cone, and comprises a projection, by which an effective surface 149 at least substantially perpendicular to the longitudinal axis is provided. The effective surface is designed to interact with the projection 142 on the laryngoscope 106. The widening 148 can be pushed onto the tube 1002 and/or may be formed by the tube 1002.

The laryngoscope 106 of FIGS. 5A and 5B is the laryngoscope of FIGS. 1A to 1D, wherein the laryngoscope comprises no rubberized surface. The lateral guide wall 126 comprises the projection 142 in the form of the step 135. As an alternative or in addition, the projection 142 may for example be formed on the tube guide wall 126, as shown in FIG. 11 and FIG. 12. In FIG. 11 and FIG. 12, the projection is designed as a wedge which extends sideways from the tube guide wall 126 into an area in which the tube 102 is guided as it is being inserted and/or pulled back. The projection 142 is designed to prevent the tube 1002 from being pulled back, by engaging behind at least part of the tube 1002, in particular the widening 148. In the case shown, the projection 142 is designed to interact with the effective surface 149 of the widening 148 in order to prevent pulling back. The tube 1002 is pushed onto the stylet 104. After insertion into the trachea, the user moves the stylet 104 and the tube 1002 sideways so that, when the stylet 104 is pulled back out of the tube 1002, the effective surface 149 of the widening 148 presses against the projection 142. While pulling the stylet back, the user continuously applies a lateral force to the tube 1002 so that the tube 1002 remains in the engaged-behind state. The projection 142 of the tube locking device 1010 may be provided as an alternative to or in addition to the step 135 (see, for example, FIG. 11 and/or FIG. 12).

As an alternative to the cone-shaped widening 148, a ring-shaped widening 148 may for example be provided on the tube 1002. For example, in FIG. 6 and FIG. 7, the tube 1002 is shown with a circumferential widening 148 which is designed as a ring. Different embodiments of widenings 148 have at least substantially similar features and barely differ in function. The projection 142 of the laryngoscope 106 can also engage behind the ring-shaped circumferential widening 148.

FIGS. 6 and 7 show a schematic representation of an intubation system 2000 in a view from below, looking toward a tube locking device 2010, and in a schematic sectional view from a proximal side. A lateral tube guide wall 2026 forms a projection 2042 which is designed to engage behind part of the tube 1002, in particular the widening 148. Furthermore, the tube locking device 2010 comprises a keeper element 144 which, by engaging behind part of the tube 1002, in particular the widening 148, is designed to prevent the tube 1002 from being pulled back. The keeper element 144 is designed as a pin 2044 which extends, at least substantially, perpendicularly from a main wall 2025 of a spatula 2008 in a manner at least substantially parallel to the tube guide wall 2026. In particular, the pin 2044 is arranged jointly with the projection 2042 in a plane perpendicular to the longitudinal direction of the tube 1002, such that the pin 2044 engages behind the widening 148 when the projection 2042 engages behind the widening 148. The keeper element 144, in particular the pin 2044, is spaced apart from the projection 2042 by a distance which at least substantially corresponds to an external diameter 2046 of the tube 1002. Proximal to the projection 2042, the tube 1002 can be guided along the tube guide wall 2026 in such a way that one side of the tube 1002 touches the tube guide wall 2026. Distally from the projection 2042, the tube guide wall 2026 is spaced apart from the tube 1002 by the projection 2042, which is designed in the manner of a step. In the region of the projection 2042, the pin 2044 is spaced apart from the projection 2042 in such a way that the tube 1002 touches the pin 2044 and the tube guide wall 2026. The pin 2044 and the projection 2042 thus define an opening. The user can insert the tube 1002 with the widening 148 into the opening in order to prevent the tube 1002 from being pulled back, and can pull the stylet 104, 109 back out of the tube 1002.

FIG. 8 shows a schematic view of another embodiment of an intubation system 3000 from below, comprising a tube locking device 3010. The intubation system 3000 is designed in a manner similar to the intubation system 100. The focus will therefore be primarily on differing features.

The tube locking device 3010 comprises a rotatable locking element 3128, which comprises a guide roller 131. The locking element 3128 is rotatable about a rotation axle 3130 which, in a pushed-forward state of the tube 102, is arranged, at least substantially, perpendicularly to a longitudinal axis 132 of the tube 102 in a region of the locking element 3128. In addition, the locking element 3128 has a locking direction of rotation and a release direction of rotation which is opposite to the locking direction of rotation. The locking element 3128 is arranged on one side of the tube guide wall 126. It comprises a bearing device, by means of which the guide roller 131 is supported. The guide roller 131 is arranged spaced apart from the side wall 126. Alternatively, the guide roller 131 may be inset into the side wall 126 or another part of the spatula 108. The locking element 3128 is arranged in a distal end region of the spatula 108. The user can press the tube 102 against the locking element 3128 when pulling the stylet 104 back out of the tube 102. When pulling the stylet back out, a force is applied to the guide roller 131 in the locking direction of rotation by way of a frictional force between the tube and the guide roller 131. In the locking direction of rotation, the guide roller 131 does not rotate or rotates only when subjected to a greater force than in the release direction of rotation. This prevents the tube 102 from being pulled out when the stylet 104 is pulled out. The guide roller rotates in the release direction of rotation, in particular when inserting the tube 102 along the spatula 108.

FIG. 16 shows a development of the intubation system 3000 in a sectional view from a distal side of the spatula 108. The tube locking device 3010 comprises a further guide roller 131, which is arranged on an opposite side in relation to the tube 102. In other words, one guide roller 131 is arranged on one side 122 of the tube 102, and one guide roller is arranged on that side 124 of the tube opposite the side 122. The tube locking device 3010 is designed to act upon the tube 102 from the two opposite sides 122, 124 of the tube 102. By virtue of the second guide roller 131, the frictional force between the tube 102 and the tube locking device 3010 can be increased, and in addition the tube 102 can be better guided during insertion into the trachea. The guide rollers 131 are spaced apart from each other by a distance which at least substantially corresponds to the external diameter of the tube 102. In the case shown, the guide rollers 131 are arranged at the side, or laterally. As an alternative or in addition, guide rollers 131 may be arranged at the top and at the bottom, i.e., on the main wall 125 and opposite the main wall 125. The tube locking device 3010 may also be designed in the manner of a tunnel, and the tube 102 may be guided through the tunnel that is formed. Alternatively, the tube locking device 3010 may define an opening, into which the tube 102 can be inserted in such a way that the guide rollers 131 can guide or hold back the tube.

Furthermore, as a development of the tube locking device of FIG. 11, a guide roller 131 may be provided in addition to the projection 142, as shown in FIG. 12. In general, a tube locking device may comprise various locking elements and/or the like. As shown in FIG. 12, the guide roller, which is similar to the guide roller of FIG. 8, may be arranged in, in particular inset into, the main wall 125. In FIG. 12, the guide roller 131 is, at least substantially, as wide as the tube 102 and extends with its width perpendicularly to the longitudinal axis 132 of the tube 102. In addition, the guide roller 131 is arranged distally from the projection 142.

FIG. 9 shows a schematic view of another embodiment of an intubation system 4000 from below, comprising a tube locking device 4010. The intubation system 4000 is designed in a manner similar to the intubation system 100. The focus will therefore be primarily on differing features.

The tube locking device 4010 comprises a rotatable locking element 4128 which comprises a clamp 4134. The clamp 4134 comprises a profiled surface, schematically shown with serrations, which is designed to interact with the tube 102. The locking element 4128 is rotatable about a rotation axle 4130 which, in a pushed-forward state of the tube 102, is arranged, at least substantially, perpendicularly to a longitudinal axis 132 of the tube 102 in a region of the locking element 4128. In addition, the locking element 4128 has a locking direction of rotation and a release direction of rotation which is opposite to the locking direction of rotation. The clamp 4134 is arranged spaced apart from the guide wall 126. The tube 102 can be guided between the clamp 4134 and the guide wall 126. When the user inserts the tube 102 (in the direction of the arrow), the clamp is loaded in the release direction of rotation, and the tube 102 can be inserted substantially without any resistance. In the opposite direction, in particular when the stylet 104 is being pulled back out of the tube 102, the clamp 4134 is loaded by the tube 102 in the locking direction of rotation. In the locking direction of rotation, the clamp 4134 is designed to rotate in particular with the profiled surface toward the tube 102. The clamp 4134 is designed to clamp the tube between the clamp 4134 and the guide wall 126, in particular when the stylet 104 is being pulled back out of the tube 102. The clamp 4134 clamps the tube 102 in particular as a result of the fact that rotation of the clamp 4134 by the tube 102 is prevented in the locking direction of rotation. The distance between at least part of the clamp 4134 and the guide wall 126 could be reduced were no tube 102 arranged. The clamping force of the clamp 4134 may steadily increase by pulling back the stylet 104. The mode of operation of the clamp 4134 is similar to that of a rope clamp, in particular a rope clamp on a sailing boat. For example, two clamps 4134 may also be provided, which move toward each other in the locking direction of rotation. As an alternative or in addition to the serrated surface which is designed to interact with the tube 102, a rubberized surface may for example be provided.

FIGS. 10A and 10B show a schematic view of another embodiment of an intubation system 5000 from below (FIG. 10A) and a sectional view of the intubation system 5000 from a proximal side (FIG. 10B), comprising a tube locking device 5010. The intubation system 5000 is designed in a manner similar to the intubation system 4000. The focus will therefore be primarily on differing features.

The tube locking device 5010 additionally comprises a tensioning element 138, which is arranged on the locking element 5128, in particular on the clamp 5134, and pre-tensions the locking element 5128, in particular the clamp 5134. The tensioning element 138 is a leg spring 140. One leg of the leg spring 140 is biased toward a lug 5131, which is formed by the laryngoscope 106. The lug 5131 extends parallel to the rotation axle 5130 of the locking element 5128. The leg spring 140 is wound with its middle portion around the rotation axle 5130 of the locking element 5128, and the second leg of the leg spring 140 is arranged against the locking element 5128 in such a way that the leg spring 140 presses the locking element 5128 in the locking direction of rotation. When the tube 102 is arranged between the locking element 5128 and the tube guide wall 126, the leg spring 140 presses the locking element 5128 against the tube and thereby pre-tensions it. A clamping force which prevents the tube 102 from being pulled back relative to the spatula 108 can thus be increased.

Locking elements, in particular rotatable locking elements as described herein, may be provided in addition to, for example, a projection. In other embodiments (not shown), a locking element may be designed to press the tube 102 against a projection in such a way that the projection engages behind at least part of the tube 102.

FIGS. 13 and 14 show a schematic view of another embodiment of an intubation system 6000 from below (FIG. 13) and a sectional view of the intubation system 6000 from a distal side (FIG. 14), comprising a tube locking device 6010. The intubation system 6000 is designed in a manner similar to the intubation system 100. The focus will therefore be primarily on differing features.

The tube locking device 6010 comprises at least one bracket 162, into which the tube 102 can be hooked. In particular, the tube 102 can be hooked sideways into the bracket 162 and can be unhooked sideways from the bracket 162. The user can hook the tube 102 into the bracket 162, in particular by moving the tube sideways. The bracket 162 comprises a bracket element 165 which is arranged on the lateral tube guide wall 126. The bracket element 165 is curved and extends along the curve at least substantially parallel to the main wall 125. The bracket 162 is designed to apply a clamping force to the tube 102. The clamping force can be applied to the tube 102 in such a way that the tube 102 can be clamped between the main wall 125 and the bracket 162. The bracket element 165 and the main wall 125 jointly define an opening 163, which opens toward the side and through which the tube 102 can be inserted sideways in order to hook the tube 102 in. The bracket 162 together with the main wall 125 and the tube guide wall 126 defines a bracket space 161, into which the tube 102 can be inserted in order to clamp the tube 102. The bracket element 165 may be bendable. In particular, the bracket element 165 may be able to be bent in such a way that the bracket space 161 is variable in size. For example, the cross-section of the bracket space 161 shown in FIG. 14 may be smaller than the cross-section of the tube 102. When the tube 102 is hooked into the bracket sideways through the opening 163, the cross-section is enlarged, and a force is applied to the tube 102 by the bracket element 165. In particular, the bracket element 165 presses the tube against the main wall 125. The curvature of the bracket element 165 may, at least substantially, correspond to the radius of the tube. In this regard, the bracket 162 may be designed in the manner of a hose clip and/or in the manner of a hose clip that is open at the side.

FIG. 15 shows an alternative embodiment of the tube locking device 6010 in a sectional view from a distal side. The bracket 162 comprises a second bracket element 165. The tube 102 can be hooked in in such a way that the bracket elements 165 clamp the tube 102 from two opposite sides of the tube 102. The remaining features, as have been described in connection with FIGS. 13 and 14, may, at least substantially, be identical.

In both alternative embodiments, the tube can be hooked sideways into the bracket 162 and can be unhooked sideways from the bracket 162. The user can hook the tube sideways into the bracket when pulling the stylet 104 back out of the tube 102. The user can thus pull the stylet 104 back on his own, using two hands. After pulling the stylet back, the user can optionally release the tube 102 by unhooking the tube 102 sideways. In this state, the user can remove the laryngoscope 106 from the patient's oral cavity, with the tube 102 remaining in the trachea.

The bracket 162 is and/or can be arranged at any advantageous position along the main direction of extension of the spatula 108. In the embodiments shown, the bracket is arranged in a distal end region of the spatula 108.

FIGS. 17A to 18B show another embodiment of an intubation system 7000. The spatula 108 and a tube 7002 jointly form a tube locking device 7010. FIGS. 17A and 17B schematically show the tube 7002 for the intubation system 7000 in a side view (FIG. 17A) and in a plan view (FIG. 17B). FIGS. 18A and 18B show the spatula 108, on which a spatula-side part of the tube locking device 7010 is arranged.

The tube 7002, in particular the tube locking device 7010, comprises a plurality of latching projections 154, in particular latching teeth 156, which each define a locked position and which are arranged one behind the other along a longitudinal axis 7022 of the tube 7002. In the side view of FIG. 17A, it can be seen that the latching projections 154 are, at least substantially, inset into the tube 7002. Furthermore, the latching projections 154, in particular the latching teeth 156, are designed in such a way that they define a continuous slope in a direction along the longitudinal axis 7022, and, as a result of the slope, widen, at least substantially, perpendicularly to the longitudinal axis 7022. In particular, the slope forms an angle of approximately 20° to 30° to the longitudinal axis 7022. It goes without saying that this is to be understood as an example, and other angles and/or geometries can also be provided. In the opposite direction, the latching projections 154, in particular latching teeth 156, each form a bearing surface 155 which is arranged, at least substantially, perpendicularly to the longitudinal axis 7022. The latching projections 154, in particular latching teeth 156, thus taper relative to the longitudinal axis 7022, in particular in a stepped manner. The latching projections 154, in particular latching teeth 156, form a sawtooth-like profile along the longitudinal axis 7022. The tube 7002 is shown in a state pushed onto the stylet 104. The arrow indicates the direction in which the stylet 104 is pulled after insertion, in order to pull the stylet 104 back out of the tube 7002 after having been pushed forward.

In FIGS. 18A and 18B, it can be seen that a retaining ring 158 is attached to the spatula 108. The retaining ring 158 is part of the tube locking device 7010. The stylet 104, together with the tube 7002 which is pushed onto the stylet 104, can be pushed forward through the retaining ring 158 relative to the spatula 108. In particular, the stylet 104 can be pushed forward together with the tube for insertion purposes. The retaining ring 158 has an internal diameter which is, at least substantially, larger than the external diameter of the tube 7002. Furthermore, the intubation system 7000 comprises at least one latching element 150, in particular a latching tongue 152. In the present case, the intubation system 7000 comprises a plurality of latching elements 150, in particular latching tongues 152. By way of example, five latching tongues 152 are arranged around the inner circumference of the retaining ring 158 and project into the opening of the retaining ring 158. The radially innermost points of the latching tongues 152 define a circle having a diameter which is smaller than the external diameter of the tube 7002. When the tube 7002 is pushed forward through the retaining ring 158 during insertion, the latching tongues 152 bend in the direction in which the tube 7002 is pushed.

The latching tongues 152 may be symmetrical in terms of bending. This may mean that the latching tongues 152 have, at least substantially, the same flexural rigidity in the direction in which the tube 7002 is pushed forward and in the opposite direction. In other embodiments, the latching tongues 152 may be asymmetrical in terms of bending. This may mean that the latching tongues 152 have a different flexural rigidity in the direction in which the tube 7002 is pushed forward than in the opposite direction. In particular, the latching tongues 152 may have a lower flexural rigidity in the direction in which the tube 7002 is pushed forward. Furthermore, a mechanical stop, which limits and/or prevents bending of the latching tongues 152, may for example be provided in the opposite direction. By way of example, the mechanical stop may be provided on the retaining ring 158 (not shown).

The at least one latching element 150, in particular the one latching tongue 152, can be moved into a released position by applying force in a first direction, in particular the direction in which the tube 7002 is pushed forward during insertion. In the released position, the latching element 150 enables the stylet 104, together with the tube 7002 which is pushed onto the stylet 104, to be pushed forward relative to the spatula 108. By applying force in a second direction, in particular when pulling the stylet 104 back out of the tube 7002, the latching element 150, in particular the latching tongue 152, can be moved into a locked position. In the locked position, the latching element 150 prevents the tube 7002 from being pulled back relative to the spatula 108. In particular, the latching element 150, by engaging behind at least one of the latching projections 154, prevents the tube 7002 from being pulled back. During insertion, the tube 7002, together with the stylet 104, is guided through the retaining ring 158. At least one of the latching tongues 152 thereby interacts with the latching teeth 156 of the tube 7002, which in particular are arranged in the manner of sawteeth. The interacting latching tongue 152 is designed to bend along the slope of the latching teeth 156 during the insertion. This means that the diameter of the circle defined by the latching tongues 152 is increased, and the tube can pass through the retaining ring 158. This means that the latching tongue 152 is in the release position. The latching tongue 152 is designed to press against the latching tooth 156, in particular radially, when the tube 7002 is being inserted. In the region in which the latching teeth 156 taper in a stepped manner, the latching tongue 152 bends back, at least substantially, into its original orientation or bending position. As a result of the latching tongue 152 being bent by the slope of the following latching tooth 156, the latching tongue is moved back into the release position. When the tube 7002 is moved in the opposite direction, the latching tongue 152 in the locked position strikes against one of the bearing surfaces 155 of the latching teeth 156. In the locked position, the latching tongue 152 is therefore, at least substantially, unable to bend. Only by applying a greater force can the tube 7002 be moved in the opposite direction. The force applied must be great enough that the latching tongue 152 bends in such a way that it no longer bears against the bearing surface 155. The mode of operation may be at least substantially similar to that of a cable tie.

FIGS. 19A to 19C show another embodiment of an intubation system 8000. The intubation system 8000 is shown in a schematic perspectival view in FIG. 19A, in a frontal view in FIG. 19B, and in a side view in FIG. 19C. Referring to all three figures, it can be seen that the intubation system 8000 comprises a laryngoscope 8006 which comprises a handle 8018 and a spatula 8008, wherein the spatula 8008 is arranged on the handle 8018. The intubation system 8000 additionally comprises a tube locking device 8010, which is designed to prevent the tube 102 from being pulled back relative to the spatula 8008 when the stylet 104 is being pulled back out of the tube 102 after having been pushed forward. The tube 102 is shown without the stylet 104. It will be understood that any tube and/or any stylet can be part of the intubation system 8000.

The tube locking device 8010 has an operating element 112, by means of which a locked state and/or a released state for the tube 102 can be established. The locked state is achieved by clamping, in particular by applying a clamping force to the tube 102. To this end, the operating element 112 has a clamping portion 114 which is located opposite the spatula 8008, in particular opposite an underside of a main wall 8025 of the spatula 8008. The underside is that side of a spatula along which a tube is guided during insertion in the case of conventional spatulas. The tube 102 can be clamped between the clamping portion 114 and the spatula 8008. In particular, the clamping force acts in such a way that the tube 102 is pressed against the spatula 8008, in particular against the underside of the main wall 8025. By means of a frictional force, the tube 102 can be prevented from being pulled back, by applying pressure to the tube 102. The clamping force can be converted into the frictional force by reference to a coefficient of friction between the tube 102 and the clamping portion 114 and/or the spatula 8008.

Furthermore, it can be seen that the clamping force can be applied to the tube 102 by operating the operating element 112. The operating element 112 can be operated by means of an operating force, wherein the operating force can be converted into the clamping force. The user applies the operating force by pulling on the operating element 112. The user can thus control the clamping force directly. If a greater clamping force is required, the user can pull harder on the operating element 112. To this end, the operating element 112 comprises a lever 116, which is pivotably arranged on the spatula 8008. The lever 116 forms the clamping portion 114 and an actuating portion 120. The lever 116 is in particular rotatably arranged on a pivot axle 8117, as a result of which it is pivotable. The pivot axle 8117 extends perpendicularly to the longitudinal direction of the tube 102 when the tube 102 is being inserted. This means that the pivot axle 8117 extends, at least substantially, perpendicularly to a main direction of extension of the spatula 8008. Furthermore, the pivot axle 8117 is arranged proximally to the tube 102, in particular closer to the handle 8018. Furthermore, the pivot axle 8117 is arranged in a proximal third of the longitudinal extent of the spatula 8008. The lever 116, in particular the clamping portion 114, extends distally from the pivot axis 8117 along the spatula 8008. In addition, the lever 116 extends in the area of the pivot axle 8117 sideways from the spatula 8008. Distally therefrom, the lever 116 has an at least substantially right-angled corner portion, which is at least in part the clamping portion 114. The tube 102 can be placed into the right-angled corner portion in such a way that the tube 102 extends between the lever 116 and the spatula 8008.

The actuating portion 120 extends in the proximal direction from the pivot axle 8117, in particular in the direction of the handle 8018 and/or at least partially along the handle 8018. As a result, the actuating portion 120 is arranged in such close proximity to the handle 8018 that the user can actuate the actuating portion 120 while gripping the handle 8018. In this embodiment, actuating means, in particular, pulling. When the user pulls the actuating portion 112 toward the handle 8018, the user clamps the tube 102 and/or establishes the locked state. If the user does not pull or pulls with relatively little force, the tube is movable, and/or the released state can be established. The actuating portion 112 extends only along a distal portion of the handle 8018. When the user grips the handle, normally only the little finger and/or the ring finger is/are located in the region of the actuating portion 112. For actuation purposes, the user uses the little finger and/or the ring finger to pull the lever 116 toward the handle 8018.

FIGS. 20A to 20C show another variant of the intubation system 8000, and for this reason the focus will be primarily on the differences. Some features are identical, as can be seen from the associated reference signs. The intubation system 8000 is shown in a schematic perspectival view in FIG. 20A, in a frontal view in FIG. 20B, and in a side view in FIG. 20C.

In this variant, an operating element 8112, in particular a lever 8116, which differs in some features is provided. The mode of operation is at least substantially identical to that of the operating element 112. The lever 8116 again comprises a clamping portion 8114 and an actuating portion 8120. The clamping portion 8114 is designed in a manner very similar to the clamping portion 114. The primary difference of the lever 8116 can be seen in the design of the actuating portion 8120. The difference is that the lever 8116 extends at least along the entire handle 8018 and/or at least partially beyond it proximally. In the region of and/or close to the handle 8018, the lever 8116 has a curvature which is designed such that the user's fingers are arranged between the lever 8112 and the handle 8018 when the user is gripping the handle 8018. The actuating portion 8120 is in particular designed to be actuated by the user's index finger when the user is gripping the handle 8018. Furthermore, the lever 8116 is spaced apart from the handle 8018 in such a way that there is sufficient space for the user's fingers when the locked state is established. The user can also grip the lever 8116 together with the handle 8018 with his hand and close his hand in such a way that the actuating portion 8120 is pivoted toward the handle 8018. As a result, the user can apply a greater operating force, which is converted by the lever 8116 into the clamping force.

FIGS. 21A and 21B show a schematic view of another embodiment of an intubation system 9000 from below (FIG. 21A) and a sectional view of the intubation system 9000 from a distal side (FIG. 21B), comprising a tube locking device 9010. The intubation system 9000 is designed in a manner similar to the intubation system 100. The focus will therefore be primarily on differing features.

The tube locking device 9010 comprises a clamping spring 164 which is designed to prevent the tube 102, by clamping it, from being pulled back. In the embodiment shown, the clamping spring 164 is a leaf spring 166. For clamping purposes, the clamping spring 164 is designed to press the tube 102 against the tube guide wall 126 by applying a lateral force. Two mounting elements 9011—in the case shown, mounting pins—are attached to the spatula 108 in such a way that they, at least substantially, project from the underside of the spatula. The tube 102 can be guided along the underside, in particular during insertion. The leaf spring 166 is attached to the mounting elements 9011 and extends, at least substantially, between the mounting elements 9011. The leaf spring 164 forms a guide surface 9013 in the region between the mounting elements 9011. The guide surface 9013 extends, at least substantially, parallel to the tube guide wall 126 and is spaced at a distance from the tube guide wall 126. The distance at least substantially corresponds to the external diameter 146 of the tube when, as in the case shown, the tube is being guided between the tube guide wall 126 and the guide surface 9013 of the leaf spring 164. The guide surface 9013 extends 3 cm along the longitudinal axis of the tube 102, as in this example, and/or is in contact with the tube along the 3 cm. The leaf spring 164 is elastically bendable perpendicularly to the longitudinal axis of the tube 102 and/or the guide surface 9013. When the tube 102 is not arranged between the leaf spring 164 and the tube guide wall 126, the guide surface 9013 is spaced from the tube guide wall 126 by a distance which is smaller than the external diameter 146 of the tube 102. As a result of the tube 102 passing between the tube guide wall 126 and the leaf spring 164, the leaf spring 164 is deformed, in particular elastically. The tube is pressed against the tube guide wall 126 by the resulting restoring force, and the tube 102 is clamped. The user can slide the tube in the longitudinal direction between the leaf spring 164 and the tube guide wall 126 in order to clamp the tube 102. As an alternative or in addition, the user can press the tube between the leaf spring 164 and the tube guide wall 126 from below, i.e., at least substantially, perpendicularly to the longitudinal direction of the tube 102. In the case of a relatively weak restoring force of the clamping spring 164, the user can guide the tube between the clamping spring 164 and the tube guide wall 126 while inserting the tube 102. In the case of a strong restoring force, the user can clamp the tube 102 between the clamping spring 164 and the tube guide wall 126 after insertion. The restoring force may for example depend upon the mechanical properties of the clamping spring 164, the external diameter 146 of the tube 102, and/or the distance between the clamping spring 164 and the tube guide wall 126.

All the intubation systems 100, 1000, 2000, 3000, 4000, 5000, 6000, 7000, 8000, 9000 described herein are designed in such a way that a stylet can be pulled back out of a tube by a single user using two hands.

### LIST OF REFERENCE SIGNS

100 intubation system
102 tube
103 cuff
104 stylet
105 balloon
106 laryngoscope
107 imaging device
108 spatula
109 video stylet
110 tube locking device
111 shaft
112 operating element
113 distal end
114 clamping portion
115 proximal end
116 lever
118 handle
120 actuating portion
122 side
124 side
125 main wall
126 tube guide wall
127 band
131 guide roller
132 longitudinal axis
135 step
136 imaging device
137 camera
138 tensioning element
140 leg spring
142 projection
144 keeper element
146 external diameter
148 widening
149 effective surface
150 latching element
152 latching tongue
154 latching projection
155 bearing surface
156 latching tooth
158 retaining ring
160 surface
161 bracket space
162 bracket
163 opening
164 clamping spring
165 bracket element
166 leaf spring
1000 intubation system
1002 tube
1010 tube locking device
2000 intubation system
2008 spatula
2010 tube locking device
2025 main wall
2026 tube guide wall
2042 projection
2044 pin
2046 external diameter
3000 intubation system
3010 tube locking device
3128 locking element
3130 rotation axle
4000 intubation system
4010 tube locking device
4128 locking element
4130 rotation axle
4134 clamp
5000 intubation system
5010 tube locking device
5128 locking element
5130 rotation axle
5131 lug
5134 clamp
6000 intubation system
6010 tube locking device
7000 intubation system
7002 tube
7010 tube locking device
7022 longitudinal axis
8000 intubation system
8006 laryngoscope
8008 spatula
8010 tube locking device
8018 handle
8025 main wall
8112 operating element
8114 clamping portion
8116 lever
8117 pivot axle
8120 actuating portion
9000 intubation system
9010 tube locking device
9011 mounting element
9013 guide surface

1. An intubation system, comprising:
a tube;
a stylet, wherein the tube can be at least partially pushed onto the stylet;
a laryngoscope, comprising a spatula, wherein, for insertion, the stylet can be pushed forward, together with the tube which is pushed onto the stylet, relative to the spatula, and wherein the stylet, after insertion, can be pulled out of the tube; and
a tube locking device, which is designed to prevent the tube from being pulled back relative to the spatula when the stylet, after having been pushed forward, is pulled back out of the tube.

2. The intubation system according to claim 1,
wherein the stylet is a video stylet which comprises an at least substantially rigid shaft, wherein the tube is configured to be pushed onto the shaft.

3. The intubation system according to claim 1,
wherein the tube locking device is modular and is configured to be attached to the spatula and removed from the spatula.

4. The intubation system according to claim 1,
wherein the spatula and/or the tube at least partially forms the tube locking device.

5. The intubation system according to claim 1,
wherein the spatula and the tube jointly form the tube locking device.

6. The intubation system according to claim 1,
wherein the tube is configured to be released by the tube locking device.

7. The intubation system according to claim 1,
wherein the tube locking device has at least one operating element, by which a locked state and/or a released state for the tube is established.

8. The intubation system according to claim 7,
wherein a clamping force is applied to the tube by operating the operating element.

9. (canceled)

10. (canceled)

11. The intubation system according to claim 7,
wherein the operating element comprises a lever which is pivotably arranged on the spatula.

12. (canceled)

13. (canceled)

14. The intubation system according to claim 1,
wherein the tube locking device is formed on one side.

15. The intubation system according to claim 1,
wherein the tube locking device is configured to act upon the tube from at least two opposite sides of the tube.

16. The intubation system according to claim 1,
wherein the spatula comprises a lateral tube guide wall; and
wherein the tube locking device is arranged on the tube guide wall.

17. The intubation system according to claim 1,
wherein the tube locking device comprises at least one rotatable locking element.

18-23. (canceled)

24. The intubation system according to claim 1,
wherein the tube locking device comprises at least one projection which is configured to prevent the tube from being pulled back, by engaging behind at least part of the tube.

25. The intubation system according to claim 1,
wherein the tube locking device comprises a keeper element which is configured to prevent the tube from being pulled back, by engaging behind at least part of the tube.

26. The intubation system at least according to claim 24,
wherein the keeper element is arranged at a distance from the projection, and
wherein the distance at least substantially corresponds to an external diameter of the tube.

27. The intubation system at least according to claim 24,
wherein the tube comprises a circumferential widening, and wherein at least part of the tube locking device can engage behind the circumferential widening.

28. The intubation system according to claim 1,
comprising at least one latching element which, when force is applied in a first direction, is moved into a released position in which the latching element allows the stylet, together with the tube which is pushed onto the stylet, to be pushed forward relative to the spatula, and which, when force is applied in a second direction, is moved into a locked position in which the latching element prevents the tube from being pulled back relative to the spatula.

29. (canceled)

30. The intubation system according to claim 1,
wherein the tube locking device comprises a retaining ring which is attached to the spatula, wherein the stylet, together with the tube which is pushed onto the stylet, is pushed forward through the retaining ring relative to the spatula.

31. The intubation system according to claim 1,
wherein the tube locking device is configured to prevent the tube from being pulled back, by applying a frictional force to the tube.

32. The intubation system according to claim 1,
wherein the tube locking device comprises at least one rubberized, profiled, and/or rough surface.

33. The intubation system according to claim 1,
wherein the tube locking device comprises at least one bracket, into which the tube is hooked.

34. (canceled)

35. The intubation system according to claim 1,
wherein the tube locking device comprises a clamping spring which is configured to prevent the tube, by clamping it, from being pulled back.

36. (canceled)

37. The intubation system according to claim 35,
wherein the clamping spring is a leaf spring.

38-40. (canceled)
